SEQUENCE LISTING (35 sequences)

SEQ ID NO: 1 - DNA, 1545 bp, Corynebacterium glutamicum
  gene (1)..(1545): glucose-6-phosphate-dehydrogenase
gtgagcacaaacacgaccccctccagctggacaaacccactgcgcgacccgcaggataaacgactcccccgcatcgctggcccttccggcatggtgatcttcggtgtcactggcgacttggctcgaaagaagctgctccccgccatttatgatctagcaaaccgcggattgctgcccccaggattctcgttggtaggttacggccgccgcgaatggtccaaagaagactttgaaaaatacgtacgcgatgccgcaagtgctggtgctcgtacggaattccgtgaaaatgtttgggagcgcctcgccgagggtatggaatttgttcgcggcaactttgatgatgatgcagctttcgacaacctcgctgcaacactcaagcgcatcgacaaaacccgcggcaccgccggcaactgggcttactacctgtccattccaccagattccttcacagcggtctgccaccagctggagcgttccggcatggctgaatccaccgaagaagcatggcgccgcgtgatcatcgagaagcctttcggccacaacctcgaatccgcacacgagctcaaccagctggtcaacgcagtcttcccagaatcttctgtgttccgcatcgaccactatttgggcaaggaaacagttcaaaacatcctggctctgcgttttgctaaccagctgtttgagccactgtggaactccaactacgttgaccacgtccagatcaccatggctgaagatattggcttgggtggacgtgctggttactacgacggcatcggcgcagcccgcgacgtcatccagaaccacctgatccagctcttggctctggttgccatggaagaaccaatttctttcgtgccagcgcagctgcaggcagaaaagatcaaggtgctctctgcgacaaagccgtgctacccattggataaaacctccgctcgtggtcagtacgctgccggttggcagggctctgagttagtcaagggacttcgcgaagaagatggcttcaaccctgagtccaccactgagacttttgcggcttgtaccttagagatcacgtctcgtcgctgggctggtgtgccgttctacctgcgcaccggtaagcgtcttggtcgccgtgttactgagattgccgtggtgtttaaagacgcaccacaccagcctttcgacggcgacatgactgtatcccttggccaaaacgccatcgtgattcgcgtgcagcctgatgaaggtgtgctcatccgcttcggttccaaggttccaggttctgccatggaagtccgtgacgtcaacatggacttctcctactcagaatccttcactgaagaatcacctgaagcatacgagcgcctcattttggatgcgctgttagatgaatccagcctcttccctaccaacgaggaagtggaactgagctggaagattctggatccaattcttgaagcatgggatgccgatggagaaccagaggattacccagcgggtacgtggggtccaaagagcgctgatgaaatgctttcccgcaacggtcacacctggcgcaggccataa

SEQ ID NO: 2 - protein, 514 aa, Corynebacterium glutamicum
  PEPTIDE (1)..(514): glucose-6-phosphate-dehydrogenase
MSTNTTPSSWTNPLRDPQDKRLPRIAGPSGMVIFGVTGDLARKKLLPAIYDLANRGLLPPGFSLVGYGRREWSKEDFEKYVRDAASAGARTEFRENVWERLAEGMEFVRGNFDDDAAFDNLAATLKRIDKTRGTAGNWAYYLSIPPDSFTAVCHQLERSGMAESTEEAWRRVIIEKPFGHNLESAHELNQLVNAVFPESSVFRIDHYLGKETVQNILALRFANQLFEPLWNSNYVDHVQITMAEDIGLGGRAGYYDGIGAARDVIQNHLIQLLALVAMEEPISFVPAQLQAEKIKVLSATKPCYPLDKTSARGQYAAGWQGSELVKGLREEDGFNPESTTETFAACTLEITSRRWAGVPFYLRTGKRLGRRVTEIAVVFKDAPHQPFDGDMTVSLGQNAIVIRVQPDEGVLIRFGSKVPGSAMEVRDVNMDFSYSESFTEESPEAYERLILDALLDESSLFPTNEEVELSWKILDPILEAWDADGEPEDYPAGTWGPKSADEMLSRNGHTWRRP

SEQ ID NO: 3 - DNA, 708 bp, Corynebacterium glutamicum
  gene (1)..(708): 6-phosphogluconolactonase
atggttgatgtagtacgcgcacgcgatactgaagatttggttgcacaggctgcctccaaattcattgaggttgttgaagcagcaactgccaataatggcaccgcacaggtagtgctcaccggtggtggcgccggcatcaagttgctggaaaagctcagcgttgatgcggctgaccttgcctgggatcgcattcatgtgttcttcggcgatgagcgcaatgtccctgtcagtgattctgagtccaatgagggccaggctcgtgaggcactgttgtccaaggtttctatccctgaagccaacattcacggatatggtctcggcgacgtagatcttgcagaggcagcccgcgcttacgaagctgtgttggatgaattcgcaccaaacggctttgatcttcacctgctcggcatgggtggcgaaggccatatcaactccctgttccctcacaccgatgcagtcaaggaatcctccgcaaaggtcatcgcggtgtttgattcccctaagcctccttcagagcgtgcaactctaacccttcctgcggttcactccgcaaagcgcgtgtggttgctggtttctggtgcggagaaggctgaggcagctgcggcgatcgtcaacggtgagcctgctgttgagtggcctgctgctggagctaccggatctgaggaaacggtattgttcttggctgatgatgctgcaggaaatctctaa

SEQ ID NO: 4 - protein, 235 aa, Corynebacterium glutamicum
  PEPTIDE (1)..(235): 6-phosphogluconolactonase
MVDVVRARDTEDLVAQAASKFIEVVEAATANNGTAQVVLTGGGAGIKLLEKLSVDAADLAWDRIHVFFGDERNVPVSDSESNEGQAREALLSKVSIPEANIHGYGLGDVDLAEAARAYEAVLDEFAPNGFDLHLLGMGGEGHINSLFPHTDAVKESSAKVIAVFDSPKPPSERATLTLPAVHSAKRVWLLVSGAEKAEAAAAIVNGEPAVEWPAAGATGSEETVLFLADDAAGNL

SEQ ID NO: 5 - DNA, 1455 bp, Corynebacterium glutamicum
  gene (1)..(1455): 6-phospho-gluconate-dehydrogenase
atgactaatggagataatctcgcacagatcggcgttgtaggcctagcagtaatgggctcaaacctcgcccgcaacttcgcccgcaacggcaacactgtcgctgtctacaaccgcagcactgacaaaaccgacaagctcatcgccgatcacggctccgaaggcaacttcatcccttctgcaaccgtcgaagagttcgtagcatccctggaaaagccacgccgcgccatcatcatggttcaggctggtaacgccaccgacgcagtcatcaaccagctggcagatgccatggacgaaggcgacatcatcatcgacggcggcaacgccctctacaccgacaccattcgtcgcgagaaggaaatctccgcacgcggtctccacttcgtcggtgctggtatctccggcggcgaagaaggcgcactcaacggcccatccatcatgcctggtggcccagcaaagtcctacgagtccctcggaccactgcttgagtccatcgctgccaacgttgacggcaccccatgtgtcacccacatcggcccagacggcgccggccacttcgtcaagatggtccacaacggcatcgagtacgccgacatgcaggtcatcggcgaggcataccaccttctccgctacgcagcaggcatgcagccagctgaaatcgctgaggttttcaaggaatggaacgcaggcgacctggattcctacctcatcgaaatcaccgcagaggttctctcccaggtggatgctgaaaccggcaagccactaatcgacgtcatcgttgacgctgcaggtcagaagggcaccggacgttggaccgtcaaggctgctcttgatctgggtattgctaccaccggcatcggcgaagctgttttcgcacgtgcactctccggcgcaaccagccagcgcgctgcagcacagggcaacctacctgcaggtgtcctcaccgatctggaagcacttggcgtggacaaggcacagttcgtcgaagacgttcgccgtgcactgtacgcatccaagcttgttgcttacgcacagggcttcgacgagatcaaggctggctccgacgagaacaactgggacgttgaccctcgcgacctcgctaccatctggcgcggcggctgcatcattcgcgctaagttcctcaaccgcatcgtcgaagcatacgatgcaaacgctgaacttgagtccctgctgctcgatccttacttcaagagcgagctcggcgacctcatcgattcatggcgtcgcgtgattgtcaccgccacccagcttggcctgccaatcccagtgttcgcttcctccctgtcctactacgacagcctgcgtgcagagcgtctgccagcagccctgatccaaggacagcgcgacttcttcggtgcgcacacctacaagcgcatcgacaaggatggctccttccacaccgagtggtccggcgaccgctccgaggttgaagcttaa

SEQ ID NO: 6 - protein, 484 aa, Corynebacterium glutamicum
  PEPTIDE (1)..(483): phospho-gluconate-dehydrogenase
MTNGDNLAQIGVVGLAVMGSNLARNFARNGNTVAVYNRSTDKTDKLIADHGSEGNFIPSATVEEFVASLEKPRRAIIMVQAGNATDAVINQLADAMDEGDIIIDGGNALYTDTIRREKEISARGLHFVGAGISGGEEGALNGPSIMPGGPAKSYESLGPLLESIAANVDGTPCVTHIGPDGAGHFVKMVHNGIEYADMQVIGEAYHLLRYAAGMQPAEIAEVFKEWNAGDLDSYLIEITAEVLSQVDAETGKPLIDVIVDAAGQKGTGRWTVKAALDLGIATTGIGEAVFARALSGATSQRAAAQGNLPAGVLTDLEALGVDKAQFVEDVRRALYASKLVAYAQGFDEIKAGSDENNWDVDPRDLATIWRGGCIIRAKFLNRIVEAYDANAELESLLLDPYFKSELGDLIDSWRRVIVTATQLGLPIPVFASSLSYYDSLRAERLPAALIQGQRDFFGAHTYKRIDKDGSFHTEWSGDRSEVEA

SEQ ID NO: 7 - DNA, 660 bp, Corynebacterium glutamicum
  gene (1)..(660): ribulose-5-phosphate epimerase
atggcacaacgtactccactaatcgccccatccattcttgctgctgatttctcccgcttaggggagcaggtgttggctgttcctgatgctgactggattcacgtcgacatcatggacggacacttcgttccaaacttgagctttggcgcggatatcacagctgcggtcaaccgcgttacggacaaagaactagacgtccacctgatgatcgaaaacccagagaagtgggtggacaactacatcgacgctggcgcggactgcattgttttccacgttgaagccaccgaaggtcacgttgagttggctaagtacatccgttccaagggtgtgcgtgcaggtttctccctgcgccctggaactcccatcgaggattacttggatgacctcgagcacttcgatgaagtcatcgtcatgagcgtcgagcctggattcggtggccaaagcttcatgcctgaacaactggaaaaggttcgtaccctgcgcaaggtcatcgatgagcgcggtctgaacaccgtcatcgagatcgacggcggcattagcgccaagaccatcaagcaggctgccgacgctggcgtggatgccttcgttgcaggttccgctgtgtacggcgctgaggatcccaacaaggcgatccaggagttgcgagcactcgcgcagtaa

SEQ ID NO: 8 - protein, 219 aa, Corynebacterium glutamicum
  PEPTIDE (1)..(219): ribulose-5-phosphate epimerase
MAQRTPLIAPSILAADFSRLGEQVLAVPDADWIHVDIMDGHFVPNLSFGADITAAVNRVTDKELDVHLMIENPEKWVDNYIDAGADCIVFHVEATEGHVELAKYIRSKGVRAGFSLRPGTPIEDYLDDLEHFDEVIVMSVEPGFGGQSFMPEQLEKVRTLRKVIDERGLNTVIEIDGGISAKTIKQAADAGVDAFVAGSAVYGAEDPNKAIQELRALAQ

SEQ ID NO: 9 - DNA, 474 bp, Corynebacterium glutamicum
  gene (1)..(474): ribose-5-phosphate isomerase
atgcgcgtataccttggagcagaccacgctggtttcgaaactaaaaatgcaatcgcagaacaccttaaggcccacggccacgaagtgatcgactgcggagcccacacctatgatgcagaagatgactacccagccttctgcatcgaagcagctagccgcacagtaaacgacccaggctcactcggcatcgtcctgggtggatccggaaacggcgagcagatcgccgccaacaaggtcaagggtgcacgttgtgcacttgcttggtctgttgaaactgcacgcctcgcccgcgagcacaacaatgcgaacctcatcggcatcggcggccgcatgcactcagaggaagaggcattggcaattgtcgacgccttcctcgagcaggaatggagcaacgccgagcgccaccagcgtcgtatcgacatcctcgctgattacgagcgcactggaatcgcacctgtcgttcctaacgaataa

SEQ ID NO: 10 - protein, 157 aa, Corynebacterium glutamicum
  PEPTIDE (1)..(157): ribose-5-phosphate isomerase
MRVYLGADHAGFETKNAIAEHLKAHGHEVIDCGAHTYDAEDDYPAFCIEAASRTVNDPGSLGIVLGGSGNGEQIAANKVKGARCALAWSVETARLAREHNNANLIGIGGRMHSEEEALAIVDAFLEQEWSNAERHQRRIDILADYERTGIAPVVPNE

SEQ ID NO: 11 - DNA, 2103 bp, Corynebacterium glutamicum
  gene (1)..(2103): transketolase
ttgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatggtgacctgcaggaaggtgtcacctctgaggcatcctccatcgctggcacccagcagctgggcaacctcatcgtgttctgggatgacaaccgcatctccatcgaagacaacactgagatcgctttcaacgaggacgttgttgctcgttacaaggcttacggctggcagaccattgaggttgaggctggcgaggacgttgcagcaatcgaagctgcagtggctgaggctaagaaggacaccaagcgacctaccttcatccgcgttcgcaccatcatcggcttcccagctccaactatgatgaacaccggtgctgtgcacggtgctgctcttggcgcagctgaggttgcagcaaccaagactgagcttggattcgatcctgaggctcacttcgcgatcgacgatgaggttatcgctcacacccgctccctcgcagagcgcgctgcacagaagaaggctgcatggcaggtcaagttcgatgagtgggcagctgccaaccctgagaacaaggctctgttcgatcgcctgaactcccgtgagcttccagcgggctacgctgacgagctcccaacatgggatgcagatgagaagggcgtcgcaactcgtaaggcttccgaggctgcacttcaggcactgggcaagacccttcctgagctgtggggcggttccgctgacctcgcaggttccaacaacaccgtgatcaagggctccccttccttcggccctgagtccatctccaccgagacctggtctgctgagccttacggccgtaacctgcacttcggtatccgtgagcacgctatgggatccatcctcaacggcatttccctccacggtggcacccgcccatacggcggaaccttcctcatcttctccgactacatgcgtcctgcagttcgtcttgcagctctcatggagaccgacgcttactacgtctggacccacgactccatcggtctgggcgaagatggcccaacccaccagcctgttgaaaccttggctgcactgcgcgccatcccaggtctgtccgtcctgcgtcctgcagatgcgaacgagaccgcccaggcttgggctgcagcacttgagtacaaggaaggccctaagggtcttgcactgacccgccagaacgttcctgttctggaaggcaccaaggagaaggctgctgaaggcgttcgccgcggtggctacgtcctggttgagggttccaaggaaaccccagatgtgatcctcatgggctccggctccgaggttcagcttgcagttaacgctgcgaaggctctggaagctgagggcgttgcagctcgcgttgtttccgttccttgcatggattggttccaggagcaggacgcagagtacatcgagtccgttctgcctgcagctgtgaccgctcgtgtgtctgttgaagctggcatcgcaatgccttggtaccgcttcttgggcacccagggccgtgctgtctcccttgagcacttcggtgcttctgcggattaccagaccctgtttgagaagttcggcatcaccaccgatgcagtcgtggcagcggccaaggactccattaacggttaa

SEQ ID NO: 12 - protein, 700 aa, Corynebacterium glutamicum
  PEPTIDE (1)..(700): transketolase
MTTLTLSPELQALTVRNYPSDWSDVDTKAVDTVRVLAADAVENCGSGHPGTAMSLAPLAYTLYQRVMNVDPQDTNWAGRDRFVLSCGHSSLTQYIQLYLGGFGLEMDDLKALRTWDSLTPGHPEYRHTKGVEITTGPLGQGLASAVGMAMAARRERGLFDPTAAEGESPFDHHIYVIASDGDLQEGVTSEASSIAGTQQLGNLIVFWDDNRISIEDNTEIAFNEDVVARYKAYGWQTIEVEAGEDVAAIEAAVAEAKKDTKRPTFIRVRTIIGFPAPTMMNTGAVHGAALGAAEVAATKTELGFDPEAHFAIDDEVIAHTRSLAERAAQKKAAWQVKFDEWAAANPENKALFDRLNSRELPAGYADELPTWDADEKGVATRKASEAALQALGKTLPELWGGSADLAGSNNTVIKGSPSFGPESISTETWSAEPYGRNLHFGIREHAMGSILNGISLHGGTRPYGGTFLIFSDYMRPAVRLAALMETDAYYVWTHDSIGLGEDGPTHQPVETLAALRAIPGLSVLRPADANETAQAWAAALEYKEGPKGLALTRQNVPVLEGTKEKAAEGVRRGGYVLVEGSKETPDVILMGSGSEVQLAVNAAKALEAEGVAARVVSVPCMDWFQEQDAEYIESVLPAAVTARVSVEAGIAMPWYRFLGTQGRAVSLEHFGASADYQTLFEKFGITTDAVVAAAKDSING

SEQ ID NO: 13 - DNA, 1083 bp, Corynebacterium glutamicum
  gene (1)..(1083): Transaldolase
atgtctcacattgatgatcttgcacagctcggcacttccacttggctcgacgacctctcccgcgagcgcattacttccggcaatctcagccaggttattgaggaaaagtctgtagtcggtgtcaccaccaacccagctattttcgcagcagcaatgtccaagggcgattcctacgacgctcagatcgcagagctcaaggccgctggcgcatctgttgaccaggctgtttacgccatgagcatcgacgacgttcgcaatgcttgtgatctgttcaccggcatcttcgagtcctccaacggctacgacggccgcgtgtccatcgaggttgacccacgtatctctgctgaccgcgacgcaaccctggctcaggccaaggagctgtgggcaaaggttgatcgtccaaacgtcatgatcaagatccctgcaaccccaggttctttgccagcaatcaccgacgctttggctgagggcatcagcgttaacgtcaccttgatcttctccgttgctcgctaccgcgaggtcatcgctgcgttcatcgagggcatcaagcaggctgctgcaaacggccacgacgtctccaagatccactctgtggcttccttcttcgtctcccgcgtcgacgttgagatcgacaagcgcctcgaggcaatcggatccgatgaggctttggctctgcgcggcaaggcaggcgttgccaacgctcagcgcgcttacgctgtgtacaaggagcttttcgacgccgccgagctgcctgaaggtgccaacactcagcgcccactgtgggcatccaccggcgtgaagaaccctgcgtacgctgcaactctttacgtttccgagctggctggtccaaacaccgtcaacaccatgccagaaggcaccatcgacgcggttctggagcagggcaacctgcacggtgacaccctgtccaactccgcggcagaagctgacgctgtgttctcccagcttgaggctctgggcgttgacttggcagatgtcttccaggtcctggagaccgagggtgtggacaagttcgttgcttcttggagcgaactgcttgagtccatggaagctcgcctgaagtag

SEQ ID NO: 14 - protein, 360 aa, Corynebacterium glutamicum
  PEPTIDE (1)..(360): Transaldolase
MSHIDDLAQLGTSTWLDDLSRERITSGNLSQVIEEKSVVGVTTNPAIFAAAMSKGDSYDAQIAELKAAGASVDQAVYAMSIDDVRNACDLFTGIFESSNGYDGRVSIEVDPRISADRDATLAQAKELWAKVDRPNVMIKIPATPGSLPAITDALAEGISVNVTLIFSVARYREVIAAFIEGIKQAAANGHDVSKIHSVASFFVSRVDVEIDKRLEAIGSDEALALRGKAGVANAQRAYAVYKELFDAAELPEGANTQRPLWASTGVKNPAYAATLYVSELAGPNTVNTMPEGTIDAVLEQGNLHGDTLSNSAAEADAVFSQLEALGVDLADVFQVLETEGVDKFVASWSELLESMEARLK

SEQ ID NO: 15 - DNA, 960 bp, Corynebacterium glutamicum
  gene (1)..(960): C. glutamicum OCPA
atgatctttgaacttccggataccaccacccagcaaatttccaagaccctaactcgactgcgtgaatcgggcacccaggtcaccaccggccgagtgctcaccctcatcgtggtcactgactccgaaagcgatgtcgctgcagttaccgagtccaccaatgaagcctcgcgcgagcacccatctcgcgtgatcattttggtggttggcgataaaactgcagaaaacaaagttgacgcagaagtccgtatcggtggcgacgctggtgcttccgagatgatcatcatgcatctcaacggacctgtcgctgacaagctccagtatgtcgtcacaccactgttgcttcctgacacccccatcgttgcttggtggccaggtgaatcaccaaagaatccttcccaggacccaattggacgcatcgcacaacgacgcatcactgatgctttgtacgaccgtgatgacgcactagaagatcgtgttgagaactatcacccaggtgataccgacatgacgtgggcgcgccttacccagtggcggggacttgttgcctcctcattggatcacccaccacacagcgaaatcacttccgtgaggctgaccggtgcaagcggcagtacctcggtggatttggctgcaggctggttggcgcggaggctgaaagtgcctgtgatccgcgaggtgacagatgctcccaccgtgccaaccgatgagtttggtactccactgctggctatccagcgcctggagatcgttcgcaccaccggctcgatcatcatcaccatctatgacgctcatacccttcaggtagagatgccggaatccggcaatgccccatcgctggtggctattggtcgtcgaagtgagtccgactgcttgtctgaggagcttcgccacatggatccagatttgggctaccagcacgcactatccggcttgtccagcgtcaagctggaaaccgtctaa

SEQ ID NO: 16 - protein, 319 aa, Corynebacterium glutamicum
  PEPTIDE (1)..(319): C. glutamicum OCPA
MIFELPDTTTQQISKTLTRLRESGTQVTTGRVLTLIVVTDSESDVAAVTESTNEASREHPSRVIILVVGDKTAENKVDAEVRIGGDAGASEMIIMHLNGPVADKLQYVVTPLLLPDTPIVAWWPGESPKNPSQDPIGRIAQRRITDALYDRDDALEDRVENYHPGDTDMTWARLTQWRGLVASSLDHPPHSEITSVRLTGASGSTSVDLAAGWLARRLKVPVIREVTDAPTVPTDEFGTPLLAIQRLEIVRTTGSIIITIYDAHTLQVEMPESGNAPSLVAIGRRSESDCLSEELRHMDPDLGYQHALSGLSSVKLETV

SEQ ID NO: 17 - protein, 445 aa, Artificial Sequence
  homoserine dehydrogenase based on Coryneform bacterium
MTSASAPSFNPGKGPGSAVGIALLGFGTVGTEVMRLMTEYGDELAHRIGGPLEVRGIAVSDISKPREGVAPELLTEDAFALIEREDVDIVVEVIGGIEYPREVVLAALKAGKSVVTANKALVAAHSAELADAAEAANVDLYFEAAVAGAIPVVGPLRRSLAGDQIQSVMGIVNGTTNFILDAMDSTGADYADSLAEATRLGYAEADPTADVEGHDAASKAAILASIAFHTRVTADDVYCEGISNISAADIEAAQQAGHTIKLLAICEKFTNKEGKSAISARVHPTLLPVSHPLASVNKSFNAIFVEAEAAGRLMFYGNGAGGAPTASAVLGDVVGAARNKVHGGRAPGESTYANLPIADFGETTTRYHLDMDVEDRVGVLAELASLFSEQGISLRTIRQEERDDDARLIVVTHSALESDLSRTVELLKAKPVVKAINSVIRLERD

SEQ ID NO: 18 - protein, 421 aa, Artificial Sequence
  aspartate kinase based on Coryneform bacterium
MALVVQKYGGSSLESAERIRNVAERIVATKKAGNDVVVVCSAMGDTTDELLELAAAVNPVPPAREMDMLLTAGERISNALVAMAIESLGAEAQSFTGSQAGVLTTERHGNARIVDVTPGRVREALDEGKICIVAGFQGVNKETRDVTTLGRGGSDTTAVALAAALNADVCEIYSDVDGVYTADPRIVPNAQKLEKLSFEEMLELAAVGSKILVLRSVEYARAFNVPLRVRSSYSNDPGTLIAGSMEDIPVEEAVLTGVATDKSEAKVTVLGISDKPGEAAKVFRALADAEINIDMVLQNVSSVEDGTTDITFTCPRSDGRRAMEILKKLQVQGNWTNVLYDDQVGKVSLVGAGMKSHPGVTAEFMEALRDVNVNIELISTSEIRISVLIREDDLDAAARALHEQFQLGGEDEAVVYAGTGR

SEQ ID NO: 19 - protein, 309 aa, Artificial Sequence
  homoserine kinase based on Coryneform bacterium
MAIELNVGRKVTVTVPGSSANLGPGFDTLGLALSVYDTVEVEIIPSGLEVEVFGEGQGEVPLDGSHLVVKAIRAGLKAADAEVPGLRVVCHNNIPQSRGLGSSAAAAVAGVAAANGLADFPLTQEQIVQLSSAFEGHPDNAAASVLGGAVVSWTNLSIDGKSQPQYAAVPLEVQDNIRATALVPNFHASTEAVRRVLPTEVTHIDARFNVSRVAVMIVALQQRPDLLWEGTRDRLHQPYRAEVLPITSEWVNRLRNRGYAAYLSGAGPTAMVLSTEPIPDKVLEDARESGIKVLELEVAGPVKVEVNQP

SEQ ID NO: 20 - DNA, 192 bp, Artificial Sequence
  promotor P3119 = PSOD, based on Coryneform bacterium
gagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttaccc

SEQ ID NO: 21 - DNA, 184 bp, Artificial Sequence
  promotor P497 = PgroES, based on Coryneform bacterium
ggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatc

SEQ ID NO: 22 - DNA, 192 bp, Artificial Sequence; identical to SEQ ID NO: 20
  promotor P1284 = PEFTU, based on Coryneform bacterium

SEQ ID NO: 23 - DNA, 114 bp, Artificial Sequence
  promotor based on Coryneform bacterium
gtcgactcatacgttaaatctatcaccgcaagggataaatatctaacaccgtgcgtgttgactattttacctctggcggtgataatggttgcatgtactaaggaggattaatta

SEQ ID NO: 24 - DNA, 7070 bp, Artificial Sequence
  Plasmid pH273 based on Coryneform bacterium
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctcctctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 25 - DNA, 7070 bp, Artificial Sequence; identical to SEQ ID NO: 24
  Plasmid pH373 based on Coryneform bacterium

SEQ ID NO: 26 - DNA, 8766 bp, Artificial Sequence
  plasmid pH304 based on Coryneform bacterium
tcgagaggcctgacgtcgggcccggtaccacgcgtcatatgactagttcggacctagggatatcgtcgacatcgatgctcttctgcgttaattaacaattgggatctctcaactaatgcagcgatgcgttctttccagaatgctttcatgacagggatgctgtcttgatcaggcaggcgtctgtgctggatgccgaagctggatttattgtcgcctttggaggtgaagttgacgctcactcgagaatcatcggccaaccatttggcattgaatgttctaggttcggaggcggaggttttctcaattagtgcgggatcgagccactgcgcccgcaggtcatcgtctccgaagagcttccacactttttcgaccggcaggttaagggttttggaggcattggccgcgaacccatcgctggtcatcccgggtttgcgcatgccacgttcgtattcataaccaatcgcgatgccttgagcccaccagccactgacatcaaagttgtccacgatgtgctttgcgatgtgggtgtgagtccaagaggtggcttttacgtcgtcaagcaattttagccactcttcccacggctttccggtgccgttgaggatagcttcaggggacatgcctggtgttgagccttgcggagtggagtcagtcatgcgaccgagactagtggcgctttgggtaccgggccccccctcgaggtcgagcggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcatgtcgacttcagttacttcaccagcccacaacaacgcacattcctccgaatttttggatgcgttggcaaaccatgtgttgatcggcgacggcgccatgggcacccagctccaaggctttgacctggacgtggaaaaggatttccttgatctggaggggtgtaatgagattctcaacgacacccgccctgatgtgttgaggcagattcaccgcgcctactttgaggcgggagctgacttggttgagaccaatacttttggttgcaacctgccgaacttggcggattatgacatcgctgatcgttgccgtgagcttgcctacaagggcactgcagtggctagggaagtggctgatgagatggggccgggccgaaacggcatgcggcgtttcgtggttggttccctgggacctggaacgaagcttccatcgctgggccatgcaccgtatgcagatttgcgtgggcactacaaggaagcagcgcttggcatcatcgacggtggtggcgatgcctttttgattgagactgctcaggacttgcttcaggtcaaggctgcggttcacggcgttcaagatgccatggctgaacttgatacattcttgcccattatttgccacgtcaccgtagagaccaccggcaccatgctcatgggttctgagatcggtgccgcgttgacagcgctgcagccactgggtatcgacatgattggtctgaactgcgccaccggcccagatgagatgagcgagcacctgcgttacctgtccaagcacgccgatattcctgtgtcggtgatgcctaacgcaggtcttcctgtcctgggtaaaaacggtgcagaatacccacttgaggctgaggatttggcgcaggcgctggctggattcgtctccgaatatggcctgtccatggtgggtggttgttgtggcaccacacctgagcacatccgtgcggtccgcgatgcggtggttggtgttccagagcaggaaacctccacactgaccaagatccctgcaggccctgttgagcaggcctcccgcgaggtggagaaagaggactccgtcgcgtcgctgtacacctcggtgccattgtcccaggaaaccggcatttccatgatcggtgagcgcaccaactccaacggttccaaggcattccgtgaggcaatgctgtctggcgattgggaaaagtgtgtggatattgccaagcagcaaacccgcgatggtgcacacatgctggatctttgtgtggattacgtgggacgagacggcaccgccgatatggcgaccttggcagcacttcttgctaccagctccactttgccaatcatgattgactccaccgagccagaggttattcgcacaggccttgagcacttgggtggacgaagcatcgttaactccgtcaactttgaagacggcgatggccctgagtcccgctaccagcgcatcatgaaactggtaaagcagcacggtgcggccgtggttgcgctgaccattgatgaggaaggccaggcacgtaccgctgagcacaaggtgcgcattgctaaacgactgattgacgatatcaccggcagctacggcctggatatcaaagacatcgttgtggactgcctgaccttcccgatctctactggccaggaagaaaccaggcgagatggcattgaaaccatcgaagccatccgcgagctgaagaagctctacccagaaatccacaccaccctgggtctgtccaatatttccttcggcctgaaccctgctgcacgccaggttcttaactctgtgttcctcaatgagtgcattgaggctggtctggactctgcgattgcgcacagctccaagattttgccgatgaaccgcattgatgatcgccagcgcgaagtggcgttggatatggtctatgatcgccgcaccgaggattacgatccgctgcaggaattcatgcagctgtttgagggcgtttctgctgccgatgccaaggatgctcgcgctgaacagctggccgctatgcctttgtttgagcgtttggcacagcgcatcatcgacggcgataagaatggccttgaggatgatctggaagcaggcatgaaggagaagtctcctattgcgatcatcaacgaggaccttctcaacggcatgaagaccgtgggtgagctgtttggttccggacagatgcagctgccattcgtgctgcaatcggcagaaaccatgaaaactgcggtggcctatttggaaccgttcatggaagaggaagcagaagctaccggatctgcgcaggcagagggcaagggcaaaatcgtcgtggccaccgtcaagggtgacgtgcacgatatcggcaagaacttggtggacatcattttgtccaacaacggttacgacgtggtgaacttgggcatcaagcagccactgtccgccatgttggaagcagcggaagaacacaaagcagacgtcatcggcatgtcgggacttcttgtgaagtccaccgtggtgatgaaggaaaaccttgaggagatgaacaacgccggcgcatccaattacccagtcattttgggtggcgctgcgctgacgcgtacctacgtggaaaacgatctcaacgaggtgtacaccggtgaggtgtactacgcccgtgatgctttcgagggcctgcgcctgatggatgaggtgatggcagaaaagcgtggtgaaggacttgatcccaactcaccagaagctattgagcaggcgaagaagaaggcggaacgtaaggctcgtaatgagcgttcccgcaagattgccgcggagcgtaaagctaatgcggctcccgtgattgttccggagcgttctgatgtctccaccgatactccaaccgcggcaccaccgttctggggaacccgcattgtcaagggtctgcccttggcggagttcttgggcaaccttgatgagcgcgccttgttcatggggcagtggggtctgaaatccacccgcggcaacgagggtccaagctatgaggatttggtggaaactgaaggccgaccacgcctgcgctactggctggatcgcctgaagtctgagggcattttggaccacgtggccttggtgtatggctacttcccagcggtcgcggaaggcgatgacgtggtgatcttggaatccccggatccacacgcagccgaacgcatgcgctttagcttcccacgccagcagcgcggcaggttcttgtgcatcgcggatttcattcgcccacgcgagcaagctgtcaaggacggccaagtggacgtcatgccattccagctggtcaccatgggtaatcctattgctgatttcgccaacgagttgttcgcagccaatgaataccgcgagtacttggaagttcacggcatcggcgtgcagctcaccgaagcattggccgagtactggcactcccgagtgcgcagcgaactcaagctgaacgacggtggatctgtcgctgattttgatccagaagacaagaccaagttcttcgacctggattaccgcggcgcccgcttctcctttggttacggttcttgccctgatctggaagaccgcgcaaagctggtggaattgctcgagccaggccgtatcggcgtggagttgtccgaggaactccagctgcacccagagcagtccacagacgcgtttgtgctctaccacccagaggcaaagtactttaacgtctaatctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 27 - DNA, 7070 bp, Artificial Sequence
  plasmid pH399 based on Coryneform bacterium
tcgagaggcctgacgtcgggcccggtaccacgcgtcatatgactagttggagaatcatgacctcagcatctgccccaagctttaaccccggcaagggtcccggctcagcagtcggaattgcccttttaggattcggaacagtcggcactgaggtgatgcgtctgatgaccgagtacggtgatgaacttgcgcaccgcattggtggcccactggaggttcgtggcattgctgtttctgatatctcaaagccacgtgaaggcgttgcacctgagctgctcactgaggacgcttttgcactcatcgagcgcgaggatgttgacatcgtcgttgaggttatcggcggcattgagtacccacgtgaggtagttctcgcagctctgaaggccggcaagtctgttgttaccgccaataaggctcttgttgcagctcactctgctgagcttgctgatgcagcggaagccgcaaacgttgacctgtacttcgaggctgctgttgcaggcgcaattccagtggttggcccactgcgtcgctccctggctggcgatcagatccagtctgtgatgggcatcgttaacggcaccaccaacttcatcttggacgccatggattccaccggcgctgactatgcagattctttggctgaggcaactcgtttgggttacgccgaagctgatccaactgcagacgtcgaaggccatgacgccgcatccaaggctgcaattttggcatccatcgctttccacacccgtgttaccgcggatgatgtgtactgcgaaggtatcagcaacatcagcgctgccgacattgaggcagcacagcaggcaggccacaccatcaagttgttggccatctgtgagaagttcaccaacaaggaaggaaagtcggctatttctgctcgcgtgcacccgactctattacctgtgtcccacccactggcgtcggtaaacaagtcctttaatgcaatctttgttgaagcagaagcagctggtcgcctgatgttctacggaaacggtgcaggtggcgcgccaaccgcgtctgctgtgcttggcgacgtcgttggtgccgcacgaaacaaggtgcacggtggccgtgctccaggtgagtccacctacgctaacctgccgatcgctgatttcggtgagaccaccactcgttaccacctcgacatggatgtggaagatcgcgtgggggttttggctgaattggctagcctgttctctgagcaaggaatcttcctgcgtacaatccgacaggaagagcgcgatgatgatgcacgtctgatcgtggtcacccactctgcgctggaatctgatctttcccgcaccgttgaactgctgaaggctaagcctgttgttaaggcaatcaacagtgtgatccgcctcgaaagggactaattttactgacatggcaattgaactgaacgtcggtcgtaaggttaccgtcacggtacctggatcttctgcaaacctcggacctggctttgacactttaggtttggcactgtcggtatacgacactgtcgaagtggaaattattccatctggcttggaagtggaagtttttggcgaaggccaaggcgaagtccctcttgatggctcccacctggtggttaaagctattcgtgctggcctgaaggcagctgacgctgaagttcctggattgcgagtggtgtgccacaacaacattccgcagtctcgtggtcttggctccgctgctgcagcggcggttgctggtgttgctgcagctaatggtttggcggatttcccgctgactcaagagcagattgttcagttgtcctctgcctttgaaggccacccagataatgctgcggcttctgtgctgggtggagcagtggtgtcgtggacaaatctgtctatcgacggcaagagccagccacagtatgctgctgtaccacttgaggtgcaggacaatattcgtgcgactgcgctggttcctaatttccacgcatccaccgaagctgtgcgccgagtccttcccactgaagtcactcacatcgatgcgcgatttaacgtgtcccgcgttgcagtgatgatcgttgcgttgcagcagcgtcctgatttgctgtgggagggtactcgtgaccgtctgcaccagccttatcgtgcagaagtgttgcctattacctctgagtgggtaaaccgcctgcgcaaccgtggctacgcggcatacctttccggtgccggcccaaccgccatggtgctgtccactgagccaattccagacaaggttttggaagatgctcgtgagtctggcattaaggtgcttgagcttgaggttgcgggaccagtcaaggttgaagttaaccaaccttaggcccaacaaggaaggcccccttcgaatcaagaagggggccttattagtgcagcaattattcgctgaacacgtgaaccttacaggtgcccggcgcgttgagtggtttgagttccagctggatgcggttgttttcaccgaggctttcttggatgaatccggcgtggatggcgcagacgaaggctgatgggcgtttgtcgttgaccacaaatgggcagctgtgtagagcgagggagtttgcttcttcggtttcggtggggtcaaagcccatttcgcggaggcggttaatgagcggggagagggcttcgtcgagttcttcggcttcggcgtggttaatgcccatgacgtgtgcccactgggttccgatggaaagtgctttggcgcggaggtcggggttgtgcattgcgtcatcgtcgacatcgccgagcatgttggccatgagttcgatcagggtgatgtattctttggcgacagcgcggttgtcggggacgcgtgtttggaagatgagggaggggcgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 28 - DNA, 6625 bp, Artificial Sequence
  pH484 based on Coryneform bacterium
tcgagaggcctgacgtcgggcccggtaccgttgctcgctgatctttcggcttaacaactttgtattcaatcagtcgggcatagaaagaaaacgcaatgatataggaaccaactgccgccaaaaccagccacacagagttgattgtttcgccacgggagaaagcgattgctccccaacccaccgccgcgataaccccaaagacaaggagaccaacgcgggcggtcggtgacattttaggggacttcttcacgcctactggaaggtcagtagcgttgctgtacaccaaatcatcgtcattgatgttgtcagtctgttttatggtcacgatctttactgttttctcttcgggtcgtttcaaagccactatgcgtagaaacagcgggcagaaacagcgggcagaaactgtgtgcagaaatgcatgcagaaaaaggaaagttcggccagatgggtgtttctgtatgccgatgatcggatctttgacagctgggtatgcgacaaatcaccgagagttgttaattcttaacaatggaaaagtaacattgagagatgatttataccatcctgcaccatttagagtggggctagtcatacccccataaccctagctgtacgcaatcgatttcaaatcagttggaaaaagtcaagaaaattacccgagacatatgcggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacatacaatgccaaagtacgacaattccaatgctgaccagtggggctttgaaacccgctccattcacgcaggccagtcagtagacgcacagaccagcgcacgaaaccttccgatctaccaatccaccgctttcgtgttcgactccgctgagcacgccaagcagcgtttcgcacttgaggatctaggccctgtttactcccgcctcaccaacccaaccgttgaggctttggaaaaccgcatcgcttccctcgaaggtggcgtccacgctgtagcgttctcctccggacaggccgcaaccaccaacgccattttgaacctggcaggagcgggcgaccacatcgtcacctccccacgcctctacggtggcaccgagactctattccttatcactcttaaccgcctgggtatcgatgtttccttcgtggaaaaccccgacgaccctgagtcctggcaggcagccgttcagccaaacaccaaagcattcttcggcgagactttcgccaacccacaggcagacgtcctggatattcctgcggtggctgaagttgcgcaccgcaacagcgttccactgatcatcgacaacaccatcgctaccgcagcgctcgtgcgcccgctcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 29 - DNA, 363 bp, Artificial Sequence
  promotor P497_P3119 = PgroES_PEFTU, based on Coryneform bacterium
cggcttaaagtttggctgccatgtgaatttttagcaccctcaacagttgagtgctggcactctcgagggtagagtgccaaataggttgtttgacacacagttgttcacccgcgacgacggctgtgctggaaacccacaaccggcacacacaaaatttttctcatggccgttaccctgcgaatgtccacagggtagctggtagtttgaaaatcaacgccgttgcccttaggattcagtaactggcacattttgtaatgcgctagatctgtgtgctcagtcttccaggctgcttatcacagtgaaagcaaaaccaattcgtggctgcgaaagtcgtagccaccacgaagtccaggaggacataca

SEQ ID NO: 30 - DNA, 6350 bp, Artificial Sequence
  plasmid pH491 based on Coryneform bacterium
tcgagctcggcgcagacgttgtcgtcgcttccctcaccaagttctacaccggcaacggctccggactgggcggcgtgcttatcgacggcggaaagttcgattggactgtcgaaaaggatggaaagccagtattcccctacttcgtcactccagatgctgcttaccacggattgaagtacgcagaccttggtgcaccagccttcggcctcaaggttcgcgttggccttctacgcgacaccggctccaccctctccgcattcaacgcatgggctgcagtccagggcatcgacaccctttccctgcgcctggagcgccacaacgaaaacgccatcaaggttgcagaattcctcaacaaccacgagaaggtggaaaaggttaacttcgcaggcctgaaggattccccttggtacgcaaccaaggaaaagcttggcctgaagtacaccggctccgttctcaccttcgagatcaagggcggcaaggatgaggcttgggcatttatcgacgccctgaagctacactccaaccttgcaaacatcggcgatgttcgctccctcgttgttcacccagcaaccaccacccattcacagtccgacgaagctggcctggcacgcgcgggcgttacccagtccaccgtccgcctgtccgttggcatcgagaccattgatgatatcatcgctgacctcgaaggcggctttgctgcaatctagcactagttcggacctagggatatcgtcgagagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgcccaccctcgcgccttcaggtcaacttgaaatccaagcgatcggtgatgtctccaccgaagccggagcaatcattacaaacgctgaaatcgcctatcaccgctggggtgaataccgcgtagataaagaaggacgcagcaatgtcgttctcatcgaacacgccctcactggagattccaacgcagccgattggtgggctgacttgctcggtcccggcaaagccatcaacactgatatttactgcgtgatctgtaccaacgtcatcggtggttgcaacggttccaccggacctggctccatgcatccagatggaaatttctggggtaatcgcttccccgccacgtccattcgtgatcaggtaaacgccgaaaaacaattcctcgacgcactcggcatcaccacggtcgccgcagtacttggtggttccatgggtggtgcccgcaccctagagtgggccgcaatgtacccagaaactgttggcgcagctgctgttcttgcagtttctgcacgcgccagcgcctggcaaatcggcattcaatccgcccaaattaaggcgattgaaaacgaccaccactggcacgaaggcaactactacgaatccggctgcaacccagccaccggactcggcgccgcccgacgcatcgcccacctcacctaccgtggcgaactagaaatcgacgaacgcttcggcaccaaagcccaaaagaacgaaaacccactcggtccctaccgcaagcccgaccagcgcttcgccgtggaatcctacttggactaccaagcagacaagctagtacagcgtttcgacgccggctcctacgtcttgctcaccgacgccctcaaccgccacgacattggtcgcgaccgcggaggcctcaacaaggcactcgaatccatcaaagttccagtccttgtcgcaggcgtagataccgatattttgtacccctaccaccagcaagaacacctctccagaaacctgggaaatctactggcaatggcaaaaatcgtatcccctgtcggccacgatgctttcctcaccgaaagccgccaaatggatcgcatcgtgaggaacttcttcagcctcatctccccagacgaagacaacccttcgacctacatcgagttctacatctaacatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 31 - DNA, 5477 bp, Artificial Sequence
  plasmid pH429 based on Coryneform bacterium
tcgagctctccaatctccactgaggtacttaatccttccggggaattcgggcgcttaaatcgagaaattaggccatcaccttttaataacaatacaatgaataattggaataggtcgacacctttggagcggagccggttaaaattggcagcattcaccgaaagaaaaggagaaccacatgcttgccctaggttggattacatggatcattattggtggtctagctggttggattgcctccaagattaaaggcactgatgctcagcaaggaattttgctgaacatagtcgtcggtattatcggtggtttgttaggcggctggctgcttggaatcttcggagtggatgttgccggtggcggcttgatcttcagcttcatcacatgtctgattggtgctgtcattttgctgacgatcgtgcagttcttcactcggaagaagtaatctgctttaaatccgtagggcctgttgatatttcgatatcaacaggccttttggtcattttggggtggaaaaagcgctagacttgcctgtggattaaaactatacgaaccggtttgtctatattggtgttagacagttcgtcgtatcttgaaacagaccaacccgaaaggacgtggccgaacgtggctgctagctaatccttgatggtggacttgctggatctcgattggtccacaacatcagtcctcttgagacggctcgcgatttggctcggcagttgttgtcggctccacctgcggactactcaatttagtttcttcattttccgaaggggtatcttcgttgggggaggcgtcgataagccccttctttttagctttaacctcagcgcgacgctgctttaagcgctgcatggcggcgcggttcatttcacgttgcgtttcgcgcctcttgttcgcgatttctttgcgggcctgttttgcttcgttgatttcggcagtacgggttttggtgagttccacgtttgttgcgtgaagcgttgaggcgttccatggggtgagaatcatcagggcgcggtttttgcgtcgtgtccacaggaagatgcgcttttctttttgttttgcgcggtagatgtcgcgctgctctaggtggtgcactttgaaatcgtcggtaagtgggtatttgcgttccaaaatgaccatcatgatgattgtttggaggagcgtccacaggttgttgctgacgcgtcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 32 - DNA, 5697 bp, Artificial Sequence
  plasmid pH449 based on Coryneform bacterium
tcgaggcgtcttccggtgtcatggttgaaccgaattccagcacaatattttccggtttaaagcaatcgatcacatagtcgattttgtccaaccactgaaaacctgcaaggaccacccaatcccctgcagcatgttcagcaaccattggcagcggcggatagcgaacttcccccttttctcccgttgccattttcgcgtcactgatcaggtgactgagctttttgtagccttccggatttttacacaagactgtcaacacgccttcttgcagactcagctccgcaccataaacggtatgcattccagcttccgcggcagcttccgcaaatctcactgcaccataaaaaccatccctatccatgactgatagagcaacaagtcctaactttttggcctgcacaaccacatcagacggatccgatgcgccagtgagaaagttataactgctggtggcatgcagctcggcaaaaggaaccgacgcttccccctgcatggcagatgaaggcgcctgcgcatccggctcatgcagcaccggacgcagagattcgacctttttacctgagaggattctttccaatttggaccacgataatggcctgccgttaaagcttcccccgccattccattccataatgataggatacatttttagaacaaattttccaataagttttccacgccagccggagaaggaaatagaccaagctgtacagatcgacgcgtcctggctgagtacaacgtcggctccggcgcagacctcaccccagttggctccagcgaaatcgtgccactggcactattctggaaggaccacgactccatcgacggcattgacggcgagtccgttgccatccctaacgatccttccaaccagggccgcgccatcaacgttctcgttcaggcaggtctggtcaccctgaagaccccaggtctggtcaccccagctccagtcgatatcgacgaggcagcttccaaggtttccgtcatcccagtcgacgcagctcaggcaccaaccgcttaccaggagggtcgcccagcgatcatcaacaactccttccttgaccgcgcaggcatcgatccaaacctcgcggtcttcgaagatgatcctgagtctgaagaagcagagccatacatcaacgtcttcgtcaccaaggctgaggacaaggacgatgccaacatcgcccgcctcgttgagctgtggcacgacccagaggttctggctgcagtagaccgcgactctgagggcacctccgtcccagttgatcgtccaggagctgaccttcaggaaatccttgatcgccttgaggctgatcaggaaaacgcataatctcttttgagttctttgcatacccatgtgcagatttctttgcacaatcacagcctgaaaatcagactgtgaacttcaaacgcatatgactagttcggacctagggatatcgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatc

SEQ ID NO: 33 - DNA, 7318 bp, Artificial Sequence
  plasmid pOM427 based on Coryneform bacterium
ggccgctcgatttaaatctcgagctctggagtgcgacaggtttgatgataaaaaattagcgcaagaagacaaaaatcaccttgcgctaatgctctgttacaggtcactaataccatctaagtagttgattcatagtgactgcatatgtaagtatttccttagataacaattgattgaatgtatgcaaataaatgcatacaccataggtgtggtttaatttgatgccctttttcagggctggaatgtgtaagagcggggttatttatgctgttgtttttttgttactcgggaagggctttacctcttccgcataaacgcttccatcagcgtttatagttaaaaaaatctttcggggggatggggagtaagcttgtgttatccgctcgggcccaatccgcaagctccaccgactcgttggcgtgcgactctagataaatatcaagcagctggccgccaataacctcagtacgcatgccacgccaagcatccctcgtgcgggccaatgcctctgcactcaaaccggaatcctgcagcatgtcttctgcccacaccaatgccatatcgccagccaaaatcgagactgaaacgccaaagtgctcgggatcgccttcgaaattattggcgcggtgatcagcttccacagcccggtgaactgtgggggctccgcgccgggtatcagaagaatcgataatatcgtcatgaatcaaggcacaagcctggatgaattcgagactcgctgcggcgtcaaggacggactcaagtttttcagaagaattcttatggccttgcgccgccaggaaaccagcccacgcataaagaggacggattcgctttcctccattgagcacgaaactgcgaagatgggccacagcatctgtgacaggagcgccgatatcagcaattgttagctcttgagcatcgaggaactgcgtcaaacgatctcgcacgacctccggaaatttgtcgaggtcaaggtcatgggcatcgaaactgctcaaggagacgtccttcaatcgaatagggggatgcgggctgaattttggtggaggtgaataaatgccagaggcagtcccaacaaaacactctcatcacactaagatacccgtcgactcatacgttaaatctatcaccgcaagggataaatatctaacaccgtgcgtgttgactattttacctctggcggtgataatggttgcatgtactaaggaggattaattaatgtccctaacgaacatcccagcctcatctcaatgggcaattagcgacgttttgaagcgtccttcacccggccgagtacctttttctgtcgagtttatgccaccccgcgacgatgcagctgaagagcgtctttaccgcgcagcagaggtcttccatgacctcggtgcatcgtttgtctccgtgacttatggtgctggcggatcaacccgtgagagaacctcacgtattgctcgacgattagcgaaacaaccgttgaccactctggtgcacctgaccctggttaaccacactcgcgaagagatgaaggcaattcttcgggaatacctagagctgggattaacaaacctgttggcgcttcgaggagatccgcctggagacccattaggcgattgggtgagcaccgatggaggactgaactatgcctctgagctcatcgatcttattaagtccactcctgagttccgggaattcgacctcggtatcgcctccttccccgaagggcatttccgggcgaaaactctagaagaagacaccaaatacactctggcgaagctgcgtggaggggcagagtactccatcacgcagatgttctttgatgtggaagactacctgcgacttcgtgatcgccggatcctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttggtacgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggc

SEQ ID NO: 34 - DNA, 5715 bp, Artificial Sequence
  plasmid pCLIK5APsodTKT based on Coryneform bacterium
cgcgtcggcaaattagtcgaatgaagttaattaaaagttcccgaatcaatctttttaatgttttcaaaccatttgaaggtgtgctgacccaggtggacgccaacctttaaaaagcttcagacttttatttccacttcataaaaactgcctgtgacgattccgttaaagattgtgccaaatcactgcgcaaaactcgcgcggaaccagaccttgccatgctatcgcctattcacactatttgagtaatcggaaatagatgggtgtagacgcttgattggcggacggttcacagcggacgatttcaggccctcgtagctcgagagtttgaaggggtccgattcgttccgttcgtgacgctttgtgaggttttttgacgttgcaccgtattgcttgccgaacatttttcttttcctttcggtttttcgagaattttcacctacaaaagcccacgtcacagctcccagacttaagattgatcacacctttgacacatttgaaccacagttggttataaaatgggttcaacatcactatggttagaggtgttgacgggtcagattaagcaaagactactttcggggtagatcacctttgccaaatttgaaccaattaacctaagtcgtagatctgatcatcggatctaacgaaaacgaaccaaaactttggtcccggtttaacccaggaaggatagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccttgaccaccttgacgctgtcacctgaacttcaggcgctcactgtacgcaattacccctctgattggtccgatgtggacaccaaggctgtagacactgttcgtgtcctcgctgcagacgctgtagaaaactgtggctccggccacccaggcaccgcaatgagcctggctccccttgcatacaccttgtaccagcgggttatgaacgtagatccacaggacaccaactgggcaggccgtgaccgcttcgttctttcttgtggccactcctctttgacccagtacatccagctttacttgggtggattcggccttgagatggatgacctgaaggctctgcgcacctgggattccttgaccccaggacaccctgagtaccgccacaccaagggcgttgagatcaccactggccctcttggccagggtcttgcatctgcagttggtatggccatggctgctcgtcgtgagcgtggcctattcgacccaaccgctgctgagggcgaatccccattcgaccaccacatctacgtcattgcttctgatgggtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

SEQ ID NO: 35 - DNA, 7506 bp, Artificial Sequence
  plasmid  pCLIK5A PSODH661 PSOD 6PGDH based on Coryneform bacterium
cgcgtcgccgaaaccgatgacagcgcggccatcggcgcccagtgcgcggtgaatgttggctagtgcaggttcgcgaccatcctcagcgagaaagcccatgacgttgccggcggagacaatgagatcaaaatcagtctctgagatctgatcaacagagagatctcccaccacccagcgaacttctggaaagtcctgcttggcgtaatcaatcaggatgggatcaaggtctgtgcctagaacatcgtggccttgcttggacaggtagccaccgatgcgtccctggccgcagccagcatccaagattttcgctcccctgggtgccatggcatcaatgaggcgggcttcgccgtaaatatcattgcctgctgcggcgaggtttcgccagcgctgcgcgtagttttctgagtgcgctgggttgttatctgtgagctctttccatgtagtcatggtgcccgagtatagggctacttgttcagcaccatggtgcgcagtgtggttcgtgcgacgacttctccgcggtgggtgcattcgatctgccacagatgggtgcggccacctagctgaatcggcgttgcttcggccacgatgacaccggagctcacagcagaaatgaagtcggtgttgttgttgatgccgacgaccatttttccaggggcggaaatcatgctggcgactgatccagtggattcggcgatggcggcgtagacaccaccgttgaccaagcccaccacttgcaggtgcttggatgccacgtgaagttcgctgaccacccggccgggctcgatggtggtgtagcgcagccccagattgcggtcgaggccataattggcgttgttgagtgcttcaagttcgtctgtggttaaagctctggtggcggcaagttctgcaagcgaaagcagatcttggggttgatcatcgcgggaagtcataattaattactctagtcggcctaaaatggttggattttcacctcctgtgacctggtaaaatcgccactacccccaaatggtcacaccttttaggccgattttgctgacaccgggcttagctgccaattattccgggcttgtgacccgctacccgataaataggtcggctgaaaaatttcgttgcaatatcaacaaaaaggcctatcattgggaggtgtcgcaccaagtacttttgcgaagcgccatctgacggattttcaaaagatgtatatgctcggtgcggaaacctacgaaaggattttttacccatgccgtcaagtacgatcaataacatgactaatggagataatctcgcacagatcggcgttgtaggcctagcagtaatgggctcaaacctcgcccgcaacttcgcccgcaacggcaacactgtcgctgtctacaaccgcagcactgacaaaaccgacaagctcatcgccgatcacggctccgaaggcaacttcatcccttctgcaaccgtcgaagagttcgtagcatccctggaaaagccacgccgcgccatcatcatggttcaggctggtaacgccaccgacgcagtcatcaaccagctggcagatgccatggacgaaggcgacatcatcatcgacggcggcaacgccctctacaccgacaccattcgtcgcgagaaggaaatctccgcacgcggtctccacttcgtcggtgctggtatctccggcggcgaagaaggcgcactcaacggcccatccatcatgcctggtggctcagcaaagtcctacgagtccctcggaccactgcttgagtccatcgctgccaacgttgacggcaccccatgtgtcacccacatcggcccagacggcgccggccacttcgtcaagatggtccacaacggcatcgagtacgccgacatgcaggtcatcggcgaggcataccaccttctccgctacgcagcaggcatgcagccagctgaaatcgctgaggttttcaaggaatggaacgcaggcgacctggattcctacctcatcgaaatcaccgcagaggttctctcccaggtggatgctgaaaccggcaagccactaatcgacgtcatcgttgacgctgcaggtcagaagggcaccggacgttggaccgtcaaggctgctcttgatctgggtattgctaccaccggcatcggcgaagctgttttcgcacgtgcactctccggcgcaaccagccagcgcgctgcagcacagggcaacctacctgcaggtgtcctcaccgatctggaagcacttggcgtggacaaggcacagttcgtcgaagacgttcgccgtgcactgtacgcatccaagcttgttgcttacgcacagggcttcgacgagatcaaggctggcttcgacgagaacaactgggacgttgaccctcgcgacctcgctaccatctggcgcggcggctgcatcattcgcgctaagttcctcaaccgcatcgtcgaagcatacgatgcaaacgctgaacttgagtccctgctgctcgatccttacttcaagagcgagctcggcgacctcatcgattcatggcgtcgcgtgattgtcaccgccacccagcttggcctgccaattccagtgttcgcttcctccctgtcctactacgacagcctgcgtgcagagcgtctgccagcagccctgatccaaggacagcgcgacttcttcggtgcgcacacctacaagcgcatcgacaaggatggctccttccacaccgagtggtccggcgaccgctccgaggttgaagcttaaaggctctccttttaacacaacgccaaaacccctcacagtcaccttagattgtgaggggtttttcgcgtgctgccagggattcgccggaggtgggcgtcgataagcaaaaatcttttaattgcttttacccatggctctgcccttgttccaataaccttgcgcgttcatgtgcgtcttgggcatgccggcgtgggtctgcagatgcttcttggccgcacgggtttcggaggattccgcgccgatccaggtataaaaatcggtgtaatccgtgtcagcgatgtgatcaatgaaggattgttcgttggaaatccactgcgcggtgatgtgctcgccctggggaaaatcgaaggtgtaatcaagtggatcgtgggcgataagatacgcggtcgcagggatttcaccgtccaaggtctccagaatcgagcagatcgctgggtaagaggtgagatcgcctaagaaaaggaagccacgcggcgctggatctgggatggcgaacggaatgtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

(19) United States
(12) Patent Application Publication
(10) Pub. No.: US 2012/0288901 A1
(43) Pub. Date: Nov. 15, 2012

(54) Method of Producing Methionine in Corynebacteria by Over-Expressing Enzymes of the Pentose Phosphate Pathway

(76) Inventors: Oskar Zelder, Speyer (DE); Hartwig Schröder, Nussloch (DE); Corinna Klopprogge, Mannheim (DE); Andrea Herold, Ketsch (DE)

(21) Appl. No.: 12/527,476

(22) PCT Filed: Feb. 13, 2008
(86) PCT No.: PCT/EP2008/051762

§ 371 Date: Aug. 17, 2009

(30) Foreign Application Priority Data
Feb. 19, 2007 (EP) 07102657.9

(51) Int. Cl. C12P 13/12; C12N 1/21

(52) U.S. Cl. 435/113; 435/252.3

(57) ABSTRACT

The present invention relates to a method of producing methionine in Coryneform bacteria in which enzymes of the pentose phosphate pathway are over-expressed. The present invention also relates to Coryneform bacteria for producing methionine in which at least two enzymes of the pentose phosphate pathway are over-expressed.

### FIELD OF THE INVENTION

The present invention relates to microorganisms and methods for producing L-methionine. In particular, the present invention relates to a method of producing methionine in Coryneform bacteria by increasing the amount and/or activity of at least one enzyme of the pentose phosphate pathway. The present invention also relates to Coryneform bacteria in which the amount and/or activity of at least two enzymes of the pentose phosphate pathway is increased.

### BACKGROUND

Currently, the worldwide annual production of methionine is about 500,000 tons. Methionine is the first limiting amino acid in livestock of poultry feed and, due to this, mainly applied as feed supplement.

In contrast to other industrial amino acids, methionine is almost exclusively applied as a racemate of D- and L-methionine which is produced by chemical synthesis. Since animals can metabolise both stereo-isomers of methionine, direct feed of the chemically produced racemic mixture is possible (D'Mello and Lewis, Effect of Nutrition Deficiencies in Animals: Amino Acids, Rechgigl (Ed.), CRC Handbook Series in Nutrition and Food, 441-490, 1978).

However, there is still a great interest in replacing the existing chemical production by a biotechnological process producing exclusively L-methionine. This is due to the fact that at lower levels of supplementation L-methionine is a better source of sulfur amino acids than D-methionine (Katz and Baker (1975) Poult. Sci. 545: 1667-74). Moreover, the chemical process uses rather hazardous chemicals and produces substantial waste streams. All these disadvantages of chemical production could be avoided by an efficient biotechnological process.

Fermentative production of fine chemicals such as amino acids, aromatic compounds, vitamins and cofactors is today typically carried out in microorganisms such as Corynebacterium glutamicum (C. glutamicum), Escherichia coli (E. coli), Saccharomyces cerevisiae (S. cerevisiae), Schizzosaccharomycs pombe (S. pombe), Pichia pastoris (P. pastoris), Aspergillus niger, Bacillus subtilis, Ashbya gossypii or Gluconobacter oxydans.

Amino acids such as glutamate are thus produced using fermentation methods. For these purposes, certain microorganisms such as Escherichia coli (E. coli) and Corynebacterium glutamicum (C. glutamicum) have proven to be particularly suitable. The production of amino acids by fermentation also has inter alia the advantage that only L-amino acids are produced and that environmentally problematic chemicals such as solvents as they are typically used in chemical synthesis are avoided.

Some attempts in the prior art to produce fine chemicals such as amino acids, lipids, vitamins or carbohydrates in microorganisms such as E. coli and C. glutamicum have tried to achieve this goal by e.g. increasing the expression of genes involved in the biosynthetic pathways of the respective fine chemicals.

Attempts to increase production of e.g. lysine by upregulating the expression of genes being involved in the biosynthetic pathway of lysine production are e.g. described in WO 02/10209, WO 2006008097, WO2005059093 or in Cremer et al. (Appl. Environ. Microbiol, (1991), 57(6), 1746-1752). However, there remains a strong need to identify further targets in metabolic pathways which can be used to beneficially influence the production of methionine in microorganisms such as C. glutamicum.

### OBJECT AND SUMMARY OF THE INVENTION

In view of this situation, it is one object of the present invention to provide Coryneform bacteria which can be used to produce L-methionine. It is a further object of the present invention to provide methods which can be used to produce L-methionine in Coryneform bacteria.

These and other objectives, as they will become apparent from the ensuing description, are solved by the present invention as described in the independent claims. The dependent claims relate to some of the preferred embodiments of the invention.

In one aspect, the invention is concerned with a method of producing L-methionine (also designated as methionine) in at least one Coryneform bacterium wherein said Coryneform bacterium is derived by genetic modification from a starting organism such that said Coryneform bacterium displays a higher amount and/or activity of at least one enzyme of the pentose phosphate pathway compared to the starting organism.

The amount and/or activity of an enzyme of the pentose phosphate pathway can be increased compared to a starting organism by increasing the copy number of nucleic acid sequences encoding said enzyme. The copy number of nucleic acid sequences encoding an enzyme of the pentose phosphate pathway can be increased using e.g. autonomously replicating vectors which comprise the nucleic acid sequences encoding said enzyme, and/or by chromosomal integration of additional copies of nucleic acid sequences encoding said enzyme into the genome of the starting organism.

An increase of the amount and/or activity of an enzyme of the pentose phosphate pathway may also be achieved by increasing transcription and/or translation of a nucleic acid sequence encoding said enzyme. An increase of transcription may be attained by use of strong promoters and/or enhancer elements. An increase in translation may be achieved if the codon usage of nucleic acid sequences encoding said enzymes is optimized for the expression in the host organism or if improved binding sites and translation initiation sites for ribosomes are installed in the upstream region of the coding sequence of a gene.

The activity of an enzyme of the pentose phosphate pathway may also be increased compared to a starting organism by introducing mutations in the genes encoding said enzymes that increase the activity of said enzymes by either shutting off negative regulatory mechanisms such as feedback inhibition or by increasing the enzymatic turnover rate of the enzyme.

In some of the preferred embodiments of the invention, the amount and/or activity of enzymes of the pentose phosphate pathway is increased compared to a starting organism by combinations of the aforementioned methods.

In one of the preferred embodiments, the invention relates to a method of producing methionine in Coryneform bacteria, wherein the amount and/or activity of at least transketolase (tkt), transaldolase (tal), glucose-6-phosphate dehydrogenase (zwf), the ocpa gene, lactonase or 6-phospho-gluconate-dehydrogenase (6PGDH) is increased compared to a starting organism.

Further preferred embodiments of the invention relate to methods for producing methionine in Coryneform bacteria, wherein the amount and/or activity of at least transketolase and 6-phospho-gluconate-dehydrogenase or glucose-6-phosphate dehydrogenase and 6-phospho-gluconate-dehydrogenase are increased compared to a starting organism.

In one of the more preferred embodiments of the invention, the amount and/or activity of transketolase and 6-phospho-gluconate-dehydrogenase is increased compared to a starting organism by replacing the respective endogenous promoters with a strong promoter, being preferably P_{SOD}. In a further elaboration of this last aspect of the invention, nucleic acid sequences are used that encode for mutated versions of transketolase, transaldolase, glucose 6-phosphate dehydrogenase, the opca protein and 6-phospho-gluconate-dehydrogenase which are either less prone to negative regulatory mechanisms and/or display a higher enzymatic turnover compared to the respective wild-type enzymes.

Another aspect of the present invention relates to a Coryneform bacterium, which is derived by genetic modification from a starting organism such that said Coryneform bacterium displays a higher amount and/or activity of at least two enzymes of the pentose phosphate pathway compared to the starting organism.

The amount and/or activity of said at least two enzymes can be increased compared to a starting organism by the aforementioned approaches, i.e. increasing the copy number of nucleic acid sequences encoding said enzymes, increasing transcription and/or translation of nucleic acid sequences encoding said enzymes and/or introducing mutations into the nucleic acid sequences encoding said enzymes which lead to more active versions of the respective enzymes.

In a preferred embodiment, the invention relates to a Coryneform bacterium in which the amount and/or activity of at least transketolase and 6-phospho-gluconate-dehydrogenase, or of at least glucose-6-phosphat-dehydrogenase and 6-phospho-gluconate-dehydrogenase is increased compared to the starting organism.

In one of the more preferred embodiments, a Coryneform bacterium is characterized in that the amount and/or activity of transketolase and 6-phospho-gluconate-dehydrogenase is increased compared to a starting organism, preferably by replacing their respective endogenous promoter with a strong promoter such as P_{SOD}.

In a further elaboration of this latter aspect of the present invention, the nucleic acid sequences of transketolase and 6-phospho-gluconate-dehydrogenase encode for mutated versions of these enzymes which are less prone to negative regulatory mechanisms and/or display a higher enzymatic turnover compared to the respective wild-type enzymes.

In all of the aforementioned embodiments of the invention, a Coryneform bacterium is selected that is preferably selected from the species of Corynebacterium glutamicum. A preferred C. glutamicum strain that can be used for the purposes of the present invention is a wild type strain such as ATCC13032 or a strain which has already been optimised for methionine production. Such latter strains will display genetic alterations such as those of DSM17322, M2014 or OM469 being described below or as being described in WO2007012078.

In one aspect of the present invention, the methods and Coryneform bacteria in accordance with the present invention allow to produce at least 2%, at least 5%, at least 10% or at least 20%, preferably at least 30%, at least 40% or at least 50%, and more preferably at least a factor of 2, at least a factor of 5 and at least a factor of 10 more methionine compared to the starting organism.

### FIGURE LEGENDS

FIG. 1 schematically depicts plasmids pCLIK int sacB PSOD TKT and pCLIK int sacB PSOD 6PGDH.

### DETAILED DESCRIPTION OF THE INVENTION

In one aspect, the present invention relates to a method of producing methionine in at least one Coryneform bacterium, wherein said Coryneform bacterium is derived by genetic modification from a starting organism such that said Coryneform bacterium displays a higher amount and/or activity of at least one enzyme of the pentose phosphate pathway compared to the starting organism.

Another embodiment of the present invention relates to a Coryneform bacterium which is derived by genetic modification from a starting organism such that said Coryneform bacterium displays a higher amount and/or activity of at least two enzymes of the pentose phosphate pathway compared to the starting organism.

It has been surprisingly been found that increasing the amount and/or activity of enzymes which are not involved directly in the metabolic pathway for methionine synthesis can lead to increased production of methionine in Coryneform bacteria. Thus, the inventors of the present invention observe that if one over-expresses at least one enzyme of the pentose phosphate pathway such as transketolase or 6-phospho-gluconate-dehydrogenase in Coryneform bacteria a higher amount of methionine is produced compared to a situation where either of these two enzymes is not expressed above their typical endogenous levels in Coryneform bacteria.

Before various aspects and some of the preferred embodiments of the invention are described in more detail, the following definitions are provided which shall have the indicated meaning throughout the description of the invention, unless explicitly indicated otherwise by the respective context.

Coryneform bacteria comprise species such as Corynebacterium glutamicum, Corynebacterium jeikeum, Corynebacterium acetoglutamicum, Corynebacterium acetoacidophilum, Corynebacterium thermoaminogenes, Corynebacterium melassecola and Corynebacterium effiziens. A preferred species is C. glutamicum.

In preferred embodiments of the invention Coryneform bacteria may be derived from the group of strains comprising C. glutamicum ATCC13032, C. glutamicum KFCC10065, C. glutamicum ATCC21608C. acetoglutamicum ATCC15806, C. acetoacidophilum ATCC13870, C. thermoaminogenes FERMBP-1539, C. melassecola ATCC17965, C. effiziens DSM 44547 and C. effiziens DSM 44549, as well as strains that are derived thereof by e.g. classical mutagenesis and selection or by directed mutagenesis.

Other particularly preferred strains of C. glutamicum may be selected from the group comprising ATCC13058, ATCC13059, ATCC13060, ATCC21492, ATCC21513, ATCC21526, ATCC21543, ATCC13287, ATCC21851, ATCC21253, ATCC21514, ATCC21516, ATCC21299, ATCC21300, ATCC39684, ATCC21488, ATCC21649, ATCC21650, ATCC19223, ATCC13869, ATCC21157, ATCC21158, ATCC21159, ATCC21355, ATCC31808, ATCC21674, ATCC21562, ATCC21563, ATCC21564, ATCC21565, ATCC21566, ATCC21567, ATCC21568, ATCC21569, ATCC21570, ATCC21571, ATCC21572, ATCC21573, ATCC21579, ATCC19049, ATCC19050, ATCC19051, ATCC19052, ATCC19053, ATCC19054, ATCC19055, ATCC19056, ATCC19057, ATCC19058, ATCC19059, ATCC19060, ATCC19185, ATCC13286, ATCC21515, ATCC21527, ATCC21544, ATCC21492, NRRL B8183, NRRL W8182, B12NRRLB12416, NRRLB12417, NRRLB12418 and NRRLB11476.

The abbreviation KFCC stands for Korean Federation of Culture Collection, ATCC stands for American-Type Strain Culture Collection and the abbreviation DSM stands for Deutsche Sammlung von Mikroorganismen. The abbreviation NRRL stands for ARS cultures collection Northern Regional Research Laboratory, Peorea, EL, USA.

For the purposes of the present invention, a preferred wild-type strain is C. glutamicum ATCC13032.

Particularly preferred are microorganisms of Corynebacterium glutamicum that are already capable of producing methionine. Therefore, strains that display genetic alterations having a similar effect such as DSM17322; M2014 or OM469 being described below are particularly preferred.

The term “starting organism” within the context of the present invention refers to a Coryneform bacterium which is used for genetic modification to increase the amount and/or activity of at least one enzyme of the penthose phosphate pathway as described below.

The terms “genetic modification” and “genetic alteration” as well as their grammatical variations within the meaning of the present invention are intended to mean that a microorganism has been modified by means of gene technology to express an altered amount of one or more proteins which can be naturally present in the respective microorganism, one or more proteins which are not naturally present in the respective microorganism, or one or more proteins with an altered activity in comparison to the proteins of the respective non-modified microorganism. A non-modified microorganism is considered to be a “starting organism”, the genetic alteration of which results in a microorganism in accordance with the present invention.

The starting organism may thus be a wild-type C. glutamicum strain such as ATCC13032.

However, the starting organism may preferably also be e.g. a C. glutamicum strain which has already been optimized for production of methionine.

Such a methionine-producing starting organism can e.g. be derived from a wild type Coryneform bacterium and preferably from a wild type C. glutamicum bacterium which contains genetic alterations in at least one of the following genes: ask^{fbr}, hom^{fbr} and metH wherein the genetic alterations lead to overexpression of any of these genes, thereby resulting in increased production of methionine relative to methionine produced in the absence of the genetic alterations. In a preferred embodiment, such a methionine producing starter organism will contain genetic alterations simulatenously in ask^{fbr}, hom^{fbr} and metH thereby resulting in increased production of methionine relative to methionine produced in the absence of the genetic alterations.

In these starting organisms, the endogenous copies of ask and horn are typically changed to feedback resisteant alleles which are no longer subject to feedback inhibition by lysine threonine, methionine or by a combination of these amino acids. This can be either done by mutation and selection or by defined genetic replacements of the genes by with mutatted alleles which code for proteins with reduced or diminished feedback inhibition. A C. glutamicum strain which includes these genetic alterations is e.g. C. glutamicum DSM17322. The person skilled in the art will be aware that alternative genetic alterations to those being described below for generation of C. glutamicum DSM17322 can be used to also achieve overexpression of ask^{fbr}, hom^{fbr} and metH.

For the purposes of the present invention, ask^{fbr} denotes a feedback resistant aspartate kinase. Hom^{fbr} denotes a feedback resistant homoserine dehydrogenase. MetH denotes a Vitamin B12-dependent methionine synthase.

In another preferred embodiment, a methionine-producing starting organism can be derived from a wild type Coryneform bacterium and preferably from a wild type C. glutamicum bacterium which contains genetic alterations in at least one of the following genes: ask^{fbr}, hom^{fbr}, metH, metA (also referred to as metX), metY (also referred to as metZ), and hsk^{mutated}. wherein the genetic alterations lead to overexpression of any of these genes, thereby resulting in increased production of methionine relative to methionine produced in the absence of the genetic alterations. In a preferred embodiment, such a methionine producing starter organism will contain genetic alterations simulatenously in ask^{fbr}, hom^{fbr}, metH, metA (also referred to as metX), metY (also referred to as metZ), and hsk^{mutated} thereby resulting in increased production of methionine relative to methionine produced in the absence of the genetic alterations.

In these starting organisms, the endogenous copies of ask, horn and hsk are typically replaced by ask^{fbr}, hom^{fbr} and hsk^{mutated} as described above for ask^{fbr} and hom^{fbr}. A C. glutamicum strain which includes these genetic alterations is e.g. C. glutamicum M2014. The person skilled in the art will be aware that alternative genetic alterations to those being described below specifically for generation of C. glutamicum M2014 can be used to also achieve overexpression of ask^{fbr}, hom^{fbr}, metH, metA (also referred to as metX), metY (also referred to as metZ), and hsk^{mutated}.

For the purposes of the present invention, metA denotes a homoserine succinyltransferase e.g. from E. coli. MetY denotes a O-Acetylhomoserine sulfhydrylase. Hsk^{mutated} denotes a homoserine kinase which has been mutated to reduce enzymatic activity. This may be achieved by exchanging threonine with serine or alanine at a position corresponding to T190 of hsk of SEQ ID No. 19. Alternatively or additionally one may replace the ATG start codon with a TTG start codon. Such mutations lead to a reduction in enzymatic activity of the resulting hsk protein compared the non-mutated hsk gene.

In another preferred embodiment, a methionine-producing starting organism can be derived from a wild type Coryneform bacterium and preferably from a wild type C. glutamicum bacterium which contains genetic alterations in at least one of the following genes: ask^{fbr}, hom^{fbr}, metH, metA (also referred to as metX), metY (also referred to as metZ), hsk^{mutated} and metF wherein the genetic alterations lead to overexpression of any of these genes, in combination with genetic alterations in at least one of the following genes: mcbR and metQ wherein the genetic alterations decrease expression of any of these genes where the combination results in increased methionine production by the microorganism relative to methionine production in absence of the combination. In a preferred embodiment, such a methionine producing starter organism will contain genetic alterations simulatenously in ask^{fbr}, hom^{fbr}, metH, metA (also referred to as metX), metY (also referred to as metZ), hsk^{mutated} and metF wherein the genetic alterations lead to overexpression of any of these genes, in combination with genetic alterations in mcbR and metQ wherein the genetic alterations decrease expression of any of these genes where the combination results in increased me thionine production by the microorganism relative to methionine production in absence of the combination.

In these starting organisms, the endogenous copies of ask, horn and hsk are typically replaced as described above while the endogenous copies of mcbR and metQ are typically functionally disrupted or deleted. A C. glutamicum strain which includes these genetic alterations is e.g. C. glutamicum OM469. The person skilled in the art will be aware that alternative genetic alterations to those being described below specifically for generation of C. glutamicum OM469 can be used to also achieve overexpression of ask^{fbr}, hom^{fbr}, metH, metA (also referred to as metX), metY (also referred to as metZ), hsk^{mutated} and metF and reduced expression of mcbR and metQ.

For the purposes of the present invention, metF denotes a N5,10-methylene-tetrahydrofolate reductase (Ec 1.5.1.20). McbR denotes a TetR-type transcriptional regulator of sulfur metabolism (Genbank accession no: AAP45010). MetQ denotes a D-methionine binding lipoprotein.

The term “enzyme of the pentose phosphate pathway” in the context of the present invention refers to the set of seven enzymes that participate in the pentose phosphate pathway according to standard textbooks. An overview of metabolic pathways such as the pentose phosphate pathway can be found at the Kyoto Encyclopedia of Genes and Genomes (http://www.genome.jp/kegg/). This database also provides overviews on species' specific modifications of metabolic pathways. For the purposes of the present invention, the following enzymes form part of the pentose phosphate pathway:
Glucose-6-phosphate-dehydrogenase (zwf, g6pdh) (EC 1.1.1.49)
6-phospho-glucono-lactonase (6 pgl) (EC 3.1.1.31)
6-phospho-gluconate-dehydrogenase (6 pgdh) (EC 1.1.1.44)
Ribulose-5-phosphate epimerase (rpe) (EC 5.1.3.1)
Ribose-5-phosphate isomerase (rpi) (EC 5.3.1.6.)
Transketolase (tkt) (EC 2.2.1.1.)
Transaldolase (tal) (EC 2.2.1.2.)

The term “increasing the amount” of at least one enzyme of the pentose phosphate pathway compared to a starting organism in the context of the present invention means that a Coryneform bacterium is genetically modified to express a higher amount of at least one of the above-mentioned enzymes of the pentose phosphate pathway. It is to be understood that increasing the amount of at least one enzyme of the pentose phosphate pathway refers to a situation where the amount of functional enzyme is increased. An enzyme of the pentose phosphate pathway in the context of the present invention is considered to be functional if it is capable of catalysing the respective reaction. There are various options to increase the amount of an enzyme in Coryneform bacteria which are well known to the person skilled in the art. These options include increasing the copy number of the nucleic acidnucleic acid sequences which encode the above-mentioned enzymes, increasing transcription and/or translation of such nucleic acid sequences. These various options will be discussed in more detail below.

The term “increasing the activity” of at least one enzyme of the pentose phosphate pathway refers to the situation that at least one mutation is introduced into the respective wild-type sequences of the above-mentioned enzyme which leads to production of more methionine compared to a situation where the same amount of wild-type enzyme is expressed. Increased production as a matter of introducing mutated versions of enzymes of the pentose phosphate pathway can be a consequence of e.g. reduced feedback inhibition. Thus, enzymes are known to reduce their catalytic activity if e.g. final product is produced by the metabolic pathway in which the enzyme participates to a sufficient degree. It is well known that one can repress such feedback inhibition by introducing, e.g. amino acid substitutions, insertions or deletions at the respective regulatory binding sites in the enzymes. Such feedback-resistant or feedback-insensitive versions of the enzyme will therefore continue to display a high activity, even when an amount of a e.g. metabolite has been produced which otherwise would down-regulate the enzyme's activity. Furthermore, the activity of an enzyme can be increased by introducing mutations which increase the catalytic turnover of an enzyme.

It is known that the enzymes of the PPP are regulated on the enzymatic level by small molecules (F Neidhardt, J L Ingraham, K B Low, B Magasanik, M Schaechter and H E Umbarger, eds. In: Escherichia coli and Salmonella typhimurium. Cellular and Molecular Biology, American Society for Microbiology, Washington, D.C. (1987). These enzymes include the Glucose-6-phosphate dehydrogenase and 6-phosphogluconate dehydrogenase which have been shown to be regulated by inhibibtion by effectors such as NADP, NADPH, ATP, fructose 1,6-bisphosphate (Fru1,6P2), D-glyceraldehyde 3-phosphate, erythrose 4-phosphate and ribulose 5-phosphate (Rib5P) and others as described in S Moritz et al (Eur. J. Biochem. (2000), 267, 3442-52) and Onishi et al. (Micorbiol. Lett. (2005), 242, 265-74). With this knowledge at hand, the skilled person can identify e.g. the binding sites for the aforementioned effectors and introduce mutations at these sites which will either increase or decrease the affinity of the enzyme for the respective regulator. Depending on the regulator's effect, the enzymativ activity can be increased.

Thus, the term “increasing the activity” of at least one enzyme refers to the situation where mutations are introduced into the wild-type sequence of any of the above-mentioned enzymes of the pentose phosphate pathway to reduce negative regulatory mechanisms such as feedback-inhibition and/or to increase catalytic turnover of the enzyme.

Of course, the approaches of increasing the amount and/or activity of at least one enzyme can be combined. Thus, it is for example possible to replace the endogenous copy of at least one enzyme of the pentose phosphate pathway in Coryneform bacteria with a mutant that encodes for the feedback-insensitive version thereof. If transcription of this mutated copy is set under the control of the strong promoter, the amount and the activity of the respective enzyme is increased. It is understood that in this case the enzyme must still be capable of catalysing the reaction in which it usually participates.

As regards the enzymes for which the amount and/or activity is to be increased in accordance with the present invention, one can use either the endogenous nucleic acid sequences of the respective Coryneform bacterium and preferably of C. glutamicum or one can use functional homologs thereof from other organisms.

Thus, one can e.g. increase the amount of glucose-6-phosphate dehydrogenase in C. glutamicum by over-expressing the respective C. glutamicum sequence, either from an autonomously replicating vector or from an additionally inserted chromosomal copy (see below) or one may use the corresponding enzymes from e.g. Bacillus subtilis or E. coli and over-express the enzyme by e.g. use of an autonomously replicable vector.

In some circumstances, it may be preferable to use the endogenous enzymes, as the endogenous coding sequence of e.g. C. glutamicum are already optimized with respect to its codon usage for expression in C. glutamicum.

In a preferred embodiment of the invention, the amount and/or activity of at least one enzyme of the pentose phosphate pathway is increased in C. glutamicum.

In a further elaboration of this aspect of the invention, one uses the respective C. glutamicum sequences to increase the amount and/or activity of at least one enzyme of the pentose phosphate pathway.

The nucleic acid sequence of C. glutamicum, glucose-6-phosphate-dehydrogenase is depicted in SEQ ID NO. 1. The corresponding amino acid sequence is depicted in SEQ ID NO. 2. The gene bank accession number (http://www.ncbi.nlm.nih.gov/) is Cg11576.

The nucleic acid sequence for 6-phosphogluconolactonase is depicted in SEQ ID NO. 3. The corresponding amino acid sequence is depicted in SEQ ID NO. 4. The gene bank accession number is Cg11578.

The nucleic acid sequence for 6-phospho-gluconate-dehydrogenase is depicted in SEQ ID NO. 5. The amino acid sequence is depicted in SEQ ID NO. 6. The gene bank accession number is Cg11452.

The nucleic acid sequence for ribulose-5-phosphate epimerase is depicted in SEQ ID NO. 7. The amino acid sequence is depicted in SEQ ID NO. 8. The gene bank accession number is Cg11598.

The nucleic acid sequence for ribose-5-phosphate isomerase is depicted in SEQ ID NO. 9. The amino acid sequence is depicted in SEQ ID NO. 10. The gene bank accession number is Cg12423.

The nucleic acid sequence for C. glutamicum transketolase is depicted in SEQ ID NO. 11. The amino acid sequence is depicted in SEQ ID NO. 12. The gene bank accession number is Cg11574.

The nucleic acid sequence of C. glutamicum transaldolase depicted in SEQ ID NO. 13. The corresponding amino acid sequence is depicted in SEQ ID NO. 14. The gene bank accession number is Cg11575.

The corresponding functional homologues to the above-mentioned C. glutamicum enzymes of the pentose phosphate pathway can be easily identified by the skilled person for other organisms by homology analyses. This can be done by determining percent identity between amino acid or nucleic acid sequences for putative homologs and the sequences for the genes or proteins encoded by them (e.g., nucleic acid sequences for transketolase, glucose-6-phosphate dehydrogenase, 6-phospho-gluconate dehydrogenase and any of the other above or below mentioned genes and proteins encoded thereby).

Percent identity may be determined, for example, by visual inspection or by using algorithm-based homology.

For example, in order to determine percent identity of two amino acid sequences, the algorithm will align the sequences for optimal comparison purposes (e.g., gaps can be introduced in the amino acid sequence of one protein for optimal alignment with the amino acid sequence of another protein). The amino acid residues at corresponding amino acid positions are then compared. When a position in one sequence is occupied by the same amino acid residue as the corresponding position in the other, then the molecules are identical at that position. The percent identity between the two sequences is a function of the number of identical positions shared by the sequences (i.e., % identity=# of identical positions/total # of positions multiplied by 100).

Various computer programs are known in the art for these purposes. For example, percent identity of two nucleic acid or amino acid sequences can be determined by comparing sequence information using the GAP computer program described by Devereux et al. (1984) Nucl. Acids. Res., 12:387 and available from the University of Wisconsin Genetics Computer Group (UWGCG). Percent identity can also be determined by aligning two nucleic acid or amino acid sequences using the Basic Local Alignment Search Tool (BLAST™) program (as described by Tatusova et al. (1999) FEMS Microbiol. Lett., 174:247.

At the filing date of this patent application, a standard software package providing the BLAST programme can be found on the BLAST website of the NCBI (http://www.ncbi.nlm.nih.gov/BLAST/). For example, if one uses any of the aforementioned SEQ IDs, one can either perform a nucleic acid sequence- or amino sequence-based BLAST search and identify closely related homologs of the respective enzymes in e.g. E. coli, S. cervisiae, Bacillus subtilis, etc. For example, for nucleic acid sequence alignments using the BLAST™ program, the default settings are as follows: reward for match is 2, penalty for mismatch is −2, open gap and extension gap penalties are 5 and 2 respectively, gap.times.dropoff is 50, expect is 10, word size is 11, and filter is OFF.

Comparable sequence searches and analysis can be performed at the EMBL database (http://www.embl.org) or the Expasy homepage (http://www.expasy.org/). All of the above sequences searches are typically performed with the default parameters as they are pre-installed by the database providers at the filing date of the present application. Homology searches may also routinely be performed using software programmes such as the laser gene software of DNA Star, Inc., Madison, Winconsin, USA, which uses the CLUSTAL method (Higgins et al. (1989), Comput. Appl. Biosci., 5(2) 151).

The skilled person understands that two proteins will likely perform the same function (e.g. provide the same enzymatic activity) if they share a certain degree of identity as described above. A typical lower limit on the amino acid level is typically at least about 25% identity. On the nucleic acid level, the lower limit is typically at least 45%.

Preferred identity grades for both type of sequences are at least about 50%, at least about 60% or least about 70%. More preferred identity levels are at least about 80%, at least about 90% or at least about 95%. These identity levels are considered to be significant.

As used herein, the terms “homology” and “homologous” are not limited to designate proteins having a theoretical common genetic ancestor, but includes proteins which may be genetically unrelated that have, none the less, evolved to perform similar functions and/or have similar structures. The requirement that the homologues should be functional means that the homologues herein described encompasse proteins that have substantially the same activity as the reference protein. For proteins to have functional homology, it is not necessarily required that they have significant identity in their amino acid sequences, but, rather, proteins having functional homology are so defined by having similar or identical activities, e.g., enzymatic activities.

Preferably, an enzyme from another organism than e.g. the host Coryneform bacteria will be considered to be a functional homolog if it shows at least significant similarity, i.e. about 50% sequence identity on the amino acid level, and catalyses the same reaction as its counterpart in the Coryneform bacterium. Functional homologues which provide the same enzymatic activity and share a higher degree of identity such as at least about 60%, at least about 70%, at least about 80% or at least about 90% sequence identity on the amino acid level are further preferred functional homolgues.

The person skilled in the art knows that one can also use fragments or mutated versions of the aforementioned enzymes from Corynefrom bacteria and of their functional homologues in other organisms as long as these fragments and mutated versions display the same type of functional activity. Typical functionally active fragments will display N-terminal and/or C-terminal deletions while mutated versions typically comprise deletions, insertions or point mutations. By way of example, a sequence of E. coli will be considered to encode for a functional homolog of C. glutamicum glucose-6-phosphate-dehydrogenase if it displays the above-mentioned identity levels on the amino acid level to SEQ ID NO. 2 and displays the same enzymatic activity. An example is the E. coli counterpart (Genbank accession number AP_{—}002472. One can also use fragments or e.g. point mutants of these sequences as long as the resulting proteins still catalyse the same type of reaction as the full-length enzymes.

According to the present invention, increasing the amount and/activity of at least one enzyme of the pentose phosphate pathway allows for improved production of methionine in Coryneform bacteria.

Improving production of methionine in Coryneform bacteria means inter alia increasing the efficiency of methionine synthesis as well as increasing the amount of methionine produced.

The term “efficiency of methionine synthesis” describes the carbon yield of methionine. This efficiency is calculated as a percentage of the energy input which entered the system in the form of a carbon substrate. Throughout the invention this value is given in percent values ((mol methio nine) (mol carbon substrate (⁻¹×100). The term “increased efficiency of methionine synthesis” thus relates to a comparison between the starting organism and the actual Coryneform bacterium in which the amount and/or activity of at least one of the enzymes of the pentose phosphate pathway has been increased.

Preferred carbon sources according to the present invention are sugars such as mono-, di- or polysaccharides. For example, sugars selected from the group comprising glucose, fructose, hanose, galactose, ribose, sorbose, lactose, maltose, sucrose, raffinose, starch or cellulose may serve as particularly preferred carbon sources.

The methods and Coryneform bacteria in accordance with the invention may also be used to produce more methionine compared to the starting organism.

The methods and Coryneform bacteria in accordance with the invention may also be used to produce methionine at a faster rate compared to the starting organism. If, for example, a typical production period is considered, the methods and Coryneform bacteria will allow to produce methionine at a faster rate, i.e. the same amount methionine will be produced at an earlier point in time compared to the starting organism. This particularly applies for the logarithmic growth phase.

Methods and Coryneform bacteria in accordance with the invention allow to produce at least about 3 g methionine/l culture volume if the strain is incubated in shake flask incubations. A titer of at least about 4 g methionine/l culture volume, at least about 5 g methionine/l culture volume or at least about 7 g methionine/l culture volume can be preferred if the strain is incubated in shake flask incubations. A more preferred value amounts to at least about 10 g methionine/l culture volume and even more preferably to at least about 20 g methionine/l cell mass if the strain is incubated in shake flask incubations.

Methods and Coryneform bacteria in accordance with the invention allow to produce at least about 25 g methionine/l culture volume if the strain is incubated in fermentation experiments using a stirred and carbon source fed fermentor. A titer of at least about 30 g methionine/l culture volume, at least about 35 g methionine/l culture volume or at least about 40 g methionine/l culture volume can be preferred if the strain is incubated in fermentation experiments using a stirred and carbon source fed fermentor. A more preferred value amounts to at least about 50 g methionine/l culture volume and even more preferably to at least about 60 g methionine/l cell mass if the strain is incubated in fermentation experiments using a stirred and carbon source fed fermentor.

In a preferred embodiment, the methods and microorganisms of the invention allow to increase the efficiency of methionine synthesis and/or the amount of methionine and/or the titer and/or the rate of methionine synthesis in comparison to the starting organism by at least about 2%, at least about 5%, at least about 10% or at least about 20%. In preferred embodiments the efficiency of methionine synthesis and/or the amount of methionine and/or the titer and/or the rate is increased compared to the starting organism by at least about 30%, at least about 40%, or at least about 50%. Even more preferred is an increase of at least about factor 2, at least about factor 3, at least about factor 5 and at least about factor 10. However, an increase of about 5% may already be considered to be a significant improvement.

According to the present invention, production of methionine in Coryneform bacteria can be improved if the amount and/or activity of at least one of the above-mentioned seven enzymes is increased in comparison to a respective starting organism.

In one aspect, it is preferred to increase the amount and/or activity of transaldolase, glucose-6-phosphate-dehydrogenase or 6-phospho-gluconate-dehydrogenase. Even more preferably, this is done in C. glutamicum.

If the amound and/or activity of glucose-6-phosphate dehydrogenase is to be increased in C. glutamicum, the skilled person will be aware that one should concomtitantly also increase the amount and/or activity of the OCPA protein for which the coding sequence is located 3′ of the gene for glucose-6-phosphate dehydrogenase in the genome in C. glutamicum. OCPA should be concomitantly overexpressed as it seems to function as a platform on which functional glucose-6-phosphate dehydrogenase is assembled (Moritz et al (vide supra)). The nucleic acid sequence of C. glutamicum OCPA depicted in SEQ ID NO. 15. The corresponding amino acid sequence is depicted in SEQ ID NO. 16. The gene bank accession number is Cg11577.

In another embodiment, the amount and/or activity of at least two enzymes of the pentose phosphate pathway is/are increased in comparison to a respective starting organism.

In one preferred embodiment, the amount and/or activity of transketolase and glucose-6-phosphate-dehydrogenase, transketolase and 6-phospho-gluconate-dehydrogenase or glucose-6-phosphate-dehydrogenase and 6-phospho-gluconate-dehydrogenase are concomitantly increased. In a further elaboration of this latter aspect, this is done in C. glutamicum.

In one aspect of the invention, it can be preferred to increase the amount and/or activity of transketolase, glucose-6-phosphate-dehydrogenase and 6-phospho-gluconate-dehydrogenase concomitantly. This can preferably be done in C. glutamicum.

If the amount and/or activity of at least four enzymes of the pentose phosphate pathway is to be increased in Coryneform bacteria, this is preferably done by concomitantly increasing the amount and/activity of transketolase, transaldolase, glucose-6-phosphate-dehydrogenase and 6-phospho-gluconate-dehydrogenase. This can preferably be done in C. glutamicum

The amount and/or activity of the above-mentioned preferred combinations of enzymes of the pentose phosphate pathway are preferably increased in C. glutamicum. To this end, one can either use a wild-type strain such as ATCC13032 or a strain carrying further genetic modifications to increase and improve methionine synthesis.

Such a strain can, for example, express a feedback-resistant homoserine dehydrogenase (hom^{fbr}). Such a strain can further express a feedback-resistant aspartate kinase (ask^{fbr}). Such a strain may additionally display increased expression of methionine synthase (metH). A strain which is suitable for production of methionine and which overeexpresses a feedback-resistant homoserine dehydrogenase, a feedback-resistant aspartate kinase and methionine synthase is e.g. the aforementioned DSM17322 of Example.

Other C. glutamicum starting strains which can be preferably used for the purposes of the present invention carry the aforementioned modifications of DSM17322 and are further optimized with respect to methionine synthesis. Such strains may for example express increased levels of a mutated homoserine kinase (hsk^{mutatedr}), a homoserine succinyltransferase (metA), and a O-Acetylhomoserine sulfhydrylase (metY) A strain which carries all these genetic alterations is e.g. M2014 of Example 1. A particularly promising starting organism in C. glutamicum for the purposes of the present invention will therefore display increased levels of metH, metY and metA, hom^{fbr}, ask^{fbr} and hsk^{mutated}.

An example of a feedback-resistant homoserine dehydrogenase carries a S393F mutation at position 393 of SEQ ID NO. 17. This hom^{fbr} shows reduced feedback inhibition by threonine and or methionine. An example of a feedback-resistant aspartate kinase carries a T311I mutation at position 311 of SEQ ID NO. 18. This ask^{fbr} shows reduced feedback inhibition by lysine and or threonine. A homoserine kinase carrying the aforementioned functional mutation carries a T190A mutation at position 190 of SEQ ID NO. 19 or a T1905 mutation at position 190 or a TTG start codon.

The C. glutamicum starting organism which may carry the aforementioned genetic alterations such as M2014 can be further improved by deleting the nucleic acid sequences for the negative regulator (mcbR) (Rey, D. et al. (2005) Mol. Microbiol., 56. 871-887, Rey, D. et al. (2003) J. Biotechnol., 103, 51-65, US2005074802) and the D-methionine binding lipoprotein (metQ) as well as by increasing expression of N5,10-methylene-tetrahydrofolate reductase (metF). A corresponding strain is described in Example 5 as OM469. Strains displaying genetic alterations that are identical to or comparable with those DSM17322, M2014 or OM469 can be preferred as C. glutamicum starting organisms.

One can increase the amount of an enzyme of the pentose phosphate pathway in a Coryneform bacterium by e.g. increasing the gene copy number, i.e. the copy number of the nucleic acid sequence encoding said enzyme, by increasing transcription, by increasing translation, and/or a combination thereof.

The person skilled in the art is familiar with the type of genetic alterations that are necessary in order to increase the gene copy number of nucleic acid sequences, to increase transcription and/or to increase translation.

In general, one can increase the copy number of a nucleic acid sequence encoding a polypeptide by expressing a vector in the Coryneform bacterium which comprises the nucleic sequence encoding said polypeptide. Such vectors can be autonomously replicable so that they can be stably kept within the Coryneform bacterium. Typical vectors for expressing polypeptides and enzymes of the pentose phosphate pathway in C. glutamicum include pCliK pB and pEKO as described in Bott, M. and Eggeling, L., eds. Handbook of Corynebacterium glutamicum. CRC Press LLC, Boca Raton, Fla.; Deb, J. K. et al. (FEMS Microbiol Lett. (1999), 175(1), 11-20), Kirchner O. et al. (J. Biotechnol. (2003), 104 (1-3), 287-299), WO2006069711 and in WO2007012078.

In another approach for increasing the copy number of nucleic acid sequences encoding a polypeptide in a Coryneform bacterium, one can integrate additional copies of nucleic acid sequences encoding such polypeptides into the chromosome of C. glutamicum. Chromosomal integration can e.g. take place at the locus where the endogenous copy of the respective polypeptide is localized. Additionally and/or alternatively, chromosomal multiplication of polypeptide encoding nucleic acid sequences can take place at other loci in the genome of a Coryneform bacterium. In case of C. glutamicum, there are various methods known to the person skilled in the art for increasing the gene copy number by chromosomal integration. One such method makes e.g. use of the vector pK19 sacB and has been described in detail in the publication of Schafer A, et al. J. Bacteriol. 1994 176(23): 7309-7319. Other vectors for chromosomal integration of polypeptide-encoding nucleic acid sequences include or pCLIK int sacB as described in WO2005059093 or WO2007011845.

Increasing the amount of at least one enzyme of the pentose phosphate pathway can also be achieved by increasing transcription of the nucleic acid sequences encoding the respective enzymes. Increased transcription will lead to more mRNA and ultimately to a higher amount of translated protein.

The person skilled in the art is aware that one can increase transcription of a coding sequence in Coryneform bacteria by numerous approaches. Thus, one can increase transcription by using strong promoters and/or strong enhancer elements. One may also use transcriptional activators such as e.g. aptamers or overexpress transcription factors. The use of strong promoters can be preferred in the context of the present invention.

A promoter is considered to be a “strong promoter” in the context of the present invention if it provides a higher degree of transcription for a nucleic acid sequence encoding a respective polypeptide than the endogenous promoter that precedes the respective nucleic acid sequence in the wild-type situation.

For the purposes of the present invention, the use of the following promoter can be considered: P_{SOD} (SEQ ID NO. 20), P_{groES} (SEQ ID NO. 21), P_{EFTu} (SEQ ID NO. 22) and ?_{pR} (SEQ ID NO. 23). These promoters are commonly used in C. glutamicum to over-express polypeptides and the strength of the promoters is considered to have the following order:

### P_{?R}>P_{EFTu}>P_{SOD}>P_{GRoES}.

The person skilled in the art is well aware that it may not always be desirable to use the strongest promoters such as ?_{PR} of the above-mentioned list. In some cases it may be necessary and sufficient to only e.g. slightly increase the amount of a first enzyme while it would be desirable to increase the amount of a second enzyme as much as possible. In such a situation, one would thus replace the endogenous promoters of the first and second enzyme in C. glutamicum with P_{EFTu} and ?_{PR}, respectively. In addition to using strong transcriptionally active promoters, choice and sequence of the so called ribosomal binding site can significantly increase the amount of an enzyme such as those described above. For example 5′ sequences adjacent to the start codon such as 15 bp upstream of the start codon influence the enzymatic activity profoundly and can be found in the sequences of P_{EFTu} (SEQ ID NO. 22), P_{groES} (SEQ ID NO. 21), P_{SOD} (SEQ ID NO. 20) and ?_{PR} (SEQ ID NO. 23).

Improvement of translation can be achieved e.g. by optimising the codon usage of the nucleic acid sequences encoding for the respective enzymes. If one uses the nucleic acid sequences of the host enzymes, adaption of the codon usage is typically not necessary but can be also applied. If however, the amount of e.g. glucose-6-phosphate-dehydrogenase (and OCPA) is to be increased by over-expression of the respective enzyme of E. coli in C. glutamicum, it may be worth considering adapting the coding sequence of the E. coli enzyme to the codon usage of C. glutamicum.

In some embodiments of the invention, it is preferred to increase the copy number of the nucleic acid sequences encoding enzymes of the pentose phosphate pathway by integrating the respective nucleic acid sequences in multiple copies at the position of the endogenous gene in the chromosome of the respective Coryneform bacterium and preferably in C. glutamicum. This approach usually preserves the genomic integrity of the genome as much as possible.

The person skilled in the art will, of course, also envisage a combination of the aforementioned approaches and thus will consider e.g. increasing the amount of glucose-6-phosphate-dehydrogenase by using the strong promoter P_{SOD} and concomitantly increasing the gene copy number for glucose-6-phosphate-dehydrogenase in C. glutamicum.

Some of the genes encoding for enzymes of the pentose phosphate pathway are organized in C. glutamicum in an operon. This operon comprises the genes for transketolase, 6-phospho-glucono-lactonase, glucose-6-phosphate-dehydrogenase and the gene called OCPA. The gene for 6-phospho-gluconate-dehydrogenase does not form part of this operon in C. glutamicum.

According to some of the above-mentioned preferred embodiments of the invention, it is preferred to increase the amount and/or activity of combinations of transketolase and 6-phospho-gluconate-dehydrogenase, transketolase and glucose-6-phosphate-dehydrogenase as well as glucose-6-phosphate-dehydrogenase and 6-phospho-gluconate-dehydrogenase. The concomitant increase of these three enzymes is also preferred.

In view of the genomic structure and location of these three enzymes in C. glutamicum, a preferred embodiment of the present invention therefore relates to methods and C. glutamicum organisms for producing methionine in which the endogenous promoter preceding the transketolase gene in C. glutamicum is replaced by a strong promoter as defined above.

In an even more preferred embodiment of the present invention, the endogenous promoter preceding transketolase in C. glutamicum is replaced with a strong promoter as defined above, and the amount and/or activity of 6-phospho-gluconate-dehydrogenase is increased as described above. Using such an approach, it is possible to achieve an increase of the amount of the enzymes transketolase, glucose-6-phosphate-dehydrogenase and optionally 6-phospho-gluconate-dehydrogenase in C. glutamicum by making minimal genetic modifications

It has further been found that one can preferably use the P_{SOD} promoter when replacing the endogenous promoter preceding the transketolase gene in C. glutamicum, as this promoter ensures efficient transcriptional activity for the purposes of increasing the amount of transketolase and the other genes of the pentose phosphate pathway operon in C. glutamicum for producing methionine. Similarly, if one increases the amount of 6-phospho-gluconate-dehydrogenase by use of a strong promoter, the P_{SOD} promoter is preferred.

In a particularly preferred embodiment, the present invention thus relates to a C. glutamicum organism in which the endogenous promoter preceding tkt in C. glutamicum is replaced by a strong promoter and in which the endogenous promoter preceding the 6-phospho-gluconate-dehydrogenase gene is replaced by a strong promoter, the strong promoter preferably being P_{SOD}.

It has been set out above that the activity of enzymes of the pentose phosphate pathway can be increased by introducing mutations in the coding sequences of these enzymes which lead e.g. to feedback-resistant versions of the respective enzymes. Specific examples for transketolase, glucose-6-phosphate-dehydrogenase and 6-phospho-gluconate-dehydrogenase will be provided below.

In case of transketolase of C. glutamicum, a mutation of alanine at a position corresponding to A293 of SEQ ID No. 12 to R and/or alanine at a position corresponding to A327 of SEQ ID No. 12 to T exchange leads to an enzyme with improved enzymatic activity. The person skilled in the art will be able to develop further or alternative mutations based on the information provided.

A particularly preferred embodiment of the present invention refers to microorganisms and methods in which the activity and amount of enzymes of the pentose phosphate pathway in C. glutamicum is increased by replacing the endogenous promoter in front of the transketolase gene of C. glutamicum with a strong promoter and preferably with the P_{SOD} promoter. In this embodiment, the transketolase may further carry a mutation providing the same effect as the aforementioned A293R and/or A327T mutation.

Alternatively and/or additionally, the glucose-6-phosphate-dehydrogenase gene may carry mutations that provide a similar effect as the above-mentioned A293R and A327T mutations for transketolase. These mutations can be but are not limited to the positions corresponding to positions 243, and/or 261, and/or 288, and/or 289, and/or 371 of SEQ ID No. 2. These positions can be mutated such that the resulting protein carries other amino acids than A243, A261, Q288, L289, V371 such as but not limited to 1^{˜}243, P261, A288, R289, A371.

In a further elaboration of this preferred embodiment of the present invention, the amount and activity of the 6-phospho-gluconate-dehydrogenase in C. glutamicum are increased. The amount is preferably increased by using a strong promoter, and preferably by P_{SOD}. The activity is increased by introducing mutations in the coding sequence of the gene for 6-phospho-gluconate-dehydrogenase that provide a similar effect as the above-mentioned A293R and A327T mutations in transketolase. In 6-phosphogluconate dehydrogenase (SEQ ID No. 6) the amino acids corresponding to positions 150, 209, 269, 288, 329, 330 and/or 353 of SEQ ID No. 6 can be mutated such that the resulting protein carries other amino acids than P150, R209, R269, A288, D329, V330, S353 such as but not limited to 150S, 209P, 269K, 288R, 329G, 330L, 353F.

The person skilled in the art knows how to introduce such point mutations into the endogenous sequences of e.g. C. glutamicum. This can e.g. be achieved by chromosomal integration of a modified nucleic acid sequence which encodes for the mutated version of e.g. transketolase into the natural locus of transketolase in C. glutamicum. Chromosomal integration at the original locus can be achieved according to the method of Schafer A, et al. J. Bacteriol. 1994 176(23): 7309-7319 and WO2007011845. One can, of course, also use e.g. sequences derived from the gene coding for E. coli transketolase which carry the mutation. In this case, the mutation should be introduced at a position corresponding to e.g. position 293 and/or 327 of SEQ ID NO. 12.

The present invention thus generally relates to methods for increasing methionine synthesis in Coryne form bacteria as well as Coryneform bacteria with increased methionine synthesis. Both aspects of the invention are characterized in that the amount and/or activity of enzymes of the pentose phosphate pathway are increased. As far as methods in accordance with the invention are concerned, the amount and/or activity of at least one enzyme of the pentose phosphate pathway is increased in Coryneform bacteria. As far as Coryneform bacteria are concerned, the invention envisages that the amount and/or activity of at least two of the enzymes of the pentose phosphate pathway are increased.

In preferred embodiments of the present invention, the amounts of enzymes of the pentose phosphate pathway are increased in C. glutamicum by replacing the endogenous promoter in front of the transketolase gene with a strong promoter which preferably is the P_{SOD} promoter. In a further development of this preferred embodiment, the amount of 6-phospho-gluconate-dehydrogenase is additionally raised, which can also be achieved by using a strong promoter. In embodiments which are even more preferred, one not only replaces the endogenous promoters in front of the transketolase gene, but one also introduces mutations into the coding sequences of the transketolase gene and optionally of the glucose-6-phosphate-dehydrogenase gene that additionally increase the activity of these enzymes. A further development of this preferred aspect of the invention includes the feature that the amount of 6-phospho-gluconate-dehydrogenase is increased in C. glutamicum by e.g. replacing the endogenous 6-phospho-gluconate-dehydrogenase promoter with a strong promoter, preferably with P_{SOD} and that the activity of 6-phospho-gluconate-dehydrogenase is increased by introducing the above-described mutations. These preferred genetic alterations can be introduced into any strain of C. glutamicum. If a wild-type strain is used, ATCC13032 can be preferred. However, in some embodiments it is preferred to use strains which are already considered to be methionine producers, such as DSM17322. Further preferred strains include the type of genetic alterations as described above, i.e. an increase of metY, metA, metH, hsk^{fbr}, ask^{fbr} and hom^{mutated} A C. glutamicum strain which carries corresponding genetic alterations is e.g. M2014. Such strains can be further improved by deletion of the mcbR regulator, down-regulation of metQ and increase of metF expression. A strain that reflects corresponding genetic alterations is OM469.

Table 1 below gives an overview on Genbank accession numbers of enzymes of the pentose phosphate pathway for different organisms. Table 2 provides Genbank accession numbers of some of the other enzymes mentioned above for different organisms.

TABLE 1
Enzymes of the pentose phosphate pathway

| Enzyme | Gene bank accession number | Organism |
|---|---|---|
| Glucose-6-phosphate- | Cgl1576, BAB98969, NCgl1514, NCgl1514, cg1778, CE1696, | Corynebacterium |
| dehydrogenase | DIP1304, jk0994, RHA1_ro07184, nfa35750, MSMEG_3101, | glutamicum and others |
| | Mmcs_2412, MAP1176c, Mb1482c, MT1494, Rv1447c, | |
| | SAV6313, Acel_1124, SCO1937, MAV_3329, Lxx11590, | |
| | BL0440, Arth_2094, Tfu_2005, itte weitere angeben | |
| OPCA protein | Cgl1577, NP_738307.1, NP_939658.1, YP_250777.1, YP_707105.1, | Corynebacterium |
| | YP_119788.1, ZP_01192082.1, NP_335942.1, ZP_01276169.1, | glutamicum and others |
| | NP_215962.1, ZP_01684361.1, YP_887415.1, | |
| | ZP_01130849.1, YP_062111.1, ZP_00615668.1, YP_953530.1, | |
| | ZP_00995403.1, YP_882512.1, NP_960109.1, YP_290062.1, | |
| | YP_831573.1, NP_827488.1, YP_947837.1, NP_822945.1, | |
| | NP_626203.1, NP_630735.1, CAH10103.1, ZP_00120910.2, | |
| | NP_695642.1, YP_909493.1, YP_872881.1, YP_923728.1, | |
| | YP_056265.1, ZP_01648612.1, ZP_01430762.1, ZP_00569428.1, | |
| | YP_714762.1, YP_480751.1, NP_301492.1, | |
| | YP_642845.1, ZP_00767699.1 | |
| 6- | Cgl1578, NCgl1516, NCgl1516, cg1780, CE1698, DIP1306, | Corynebacterium |
| phosphogluconolactonase | Mmcs_2410, MSMEG_3099, Mb1480c, MT1492, Rv1445c, | glutamicum and others |
| | MAV_3331, RHA1_ro07182, nfa35770, MAP1174c, ML0579, | |
| | jk0996, Tfu_2007, FRAAL4578, SAV6311, SCO1939, SCC22.21, | |
| | TW464 | |
| 6-phospho-gluconate- | Cgl1452, BAB98845, NCgl1396, cgl1452, NCgl1396, cg1643, | Corynebacterium |
| dehydrogenase | DIP1213, CE1588, jk0912, RHA1_ro07246, nfa11750, Mmcs_2812, | glutamicum and others |
| | MSMEG_3632, MT1892, Rv1844c, MAV_2871, | |
| | MAP1557c, ML2065, SAV724, SCO0975, SCBAC19F3.02, | |
| | BL0444, Lxx17380, Arth_2449, Mb1875c, OB0185 | |
| | Bitte weitere angeben | |
| Ribulose-5-P-epimerase | Cgl1598, cg1801, CE1717, DIP1320, MSMEG_3066, Mb1443, | Corynebacterium |
| | MT1452, Rv1408, MAV_3370, ML0554, jk1011, MAP1135, | glutamicum and others |
| | RHA1_ro07167, Mmcs_2385, nfa36030, SCO1464, | |
| | SAV6880, FRAAL5223, Acel_1276, BL0753 | |
| Ribose-5-P-isomerase | Cgl2423, cg2658, CE2318, DIP1796, nfa13270, jk0541, RHA1_ro01378, | Corynebacterium |
| | MSMEG_4684, Mmcs_3599, Mb2492c, Rv2465c, | glutamicum and others |
| | MT2540, ML1484, MAV_1707, MAP2285c, SCO2627, | |
| | SAV5426, Tfu_2202, Arth_2408, PPA1624, Francci3_1162 | |
| Transketolase | Cgl1574, YP_225858, cg1774, CE1694, DIP1302, jk0992, nfa35730, | Corynebacterium |
| | RHA1_ro07186, MSMEG_3103, MAP1178c, ML0583, | glutamicum and others |
| | MAV_3327, Mb1484c, MT1496, Rv1449c, Mmcs_2414, | |
| | Tfu_2002, Arth_2097, Lxx11620, SAV1766, SCO1935, Acel_1127 | |
| Transaldolase | Cgl1575, cg1776, CE1695, DIP1303, jk0993, Mmcs_2413, | Corynebacterium |
| | MSMEG_3102, MAP1177c, RHA1_ro07185, MAV_3328, | glutamicum and others |
| | Mb1483c, Rv1448c, MT1495, nfa35740, ML0582, Arth_2096, | |
| | Lxx11610, SAV1767, Tfu_2003, SCO1936, Francci3_1648 | |

TABLE 2
enzymes of methionine producing organisms

| Enzyme | Gene bank accession number | Organism |
|---|---|---|
| Methylene | Cgl2171, CE2066, cg2383, DIP1611, jk0737, RHA1_ro01105, nfa17400, | C. glutamicum and |
| tetrahydrofolate | Tfu_1050, Acel_0991, SAV6100, SCO2103, FRAAL2163, Francci3_1389, | others |
| reductase (metF) | aq_1429, TTC1656, TTHA0327, ELI_10095, CT1368, | |
| | Sala_0035, DP1612, Pcar_1732 | |
| cob(I)alamin | Cgl1507, CE1637, cg1701, DIP1259, RHA1_ro00859, nfa31930, Rv2124c, | C. glutamicum and |
| dependent | Mb2148c, ML1307, SCO1657, Tfu_1825, SAV6667, Arth_3627, | others |
| methionine | Acel_1174, MT2183, GOX2074, tll1027, GbCGDNIH1_0151, | |
| synthase (metH) | Rru_A1531, alr0308, slr0212 | |
| O- | Cgl0653, NCgl0625, cg0755, CE0679, DIP0630, jk1694, | C. glutamicum and |
| acetylhomoserine | MAP3457, Mb3372, MT3443, Rv3340, nfa35960, Lxx18930, | others |
| sulfhydrolase | Tfu_2823, CAC2783, GK0284, BH2603, lmo0595, lin0604, | |
| (metY) | LMOf2365_0624, ABC0432, TTE2151, BT2387, STH2782, | |
| | str0987, stu0987, BF1406, SH0593, BF1342, lp_2536, L75975, | |
| | OB3048, BL0933, LIC11852, LA2062, BMAA1890, | |
| | BPSS0190, SMU.1173, BB1055, PP2528, PA5025, | |
| | PBPRB1415, GSU1183, RPA2763, WS1015, TM0882, | |
| | VP0629, BruAb1_0807, BMEI1166, BR0793, CPS_2546, | |
| | XC_1090, XCC3068, plu3517, PMT0875, SYNW0851, | |
| | Pro0800, CT0604, NE1697, RB8221, bll1235, syc1143_c, | |
| | ACIAD3382, ebA6307, RSc1562, Daro_2851, DP2506, | |
| | DR0873, MA2715, PMM0642, PMN2A_0083, IL2014, | |
| | SPO1431, ECA0820, AGR_C_2311, Atu1251, mlr8465, | |
| | SMc01809, CV1934, SPBC428.11, PM0738, SO1095, | |
| | SAR11_1030, PFL_0498, CTC01153, BA_0514, BCE5535, | |
| | BAS5258, GBAA5656, BA5656, BCZK5104, TTHA0760, | |
| | TTC0408, BC5406, BT9727_5087, HH0636, YLR303W, | |
| | ADL031W, CJE1895, spr1095, rrnAC2716, orf19.5645, | |
| | Cj1727c, VNG2421G, PSPPH_1663, XOO1390, Psyr_1669, | |
| | PSPTO3810, MCA2488, TDE2200, FN1419, PG0343, | |
| | Psyc_0792, MS1347, CC3168, Bd3795, MM3085, 389.t00003, | |
| | NMB1609, SAV3305, NMA1808, GOX1671, APE1226, | |
| | XAC3602, NGO1149, ZMO0676, SCO4958, lpl0921, lpg0890, | |
| | lpp0951, EF0290, BPP2532, CBU2025, BP3528, BLi02853, | |
| | BL02018, BG12291, CG5345-PA, HP0106, ML0275, jhp0098, | |
| | At3g57050, 107869, HI0086, NTHI0100, SpyM3_0133, | |
| | SPs0136, spyM18_0170, M6_Spy0192, SE2323, SERP0095, | |
| | SPy0172, PAB0605, DDB0191318, ST0506, F22B8.6, | |
| | PTO1102, CPE0176, PD1812, XF0864, SAR0460, | |
| | SACOL0503, SA0419, Ta0080, PF1266, MW0415, SAS0418, | |
| | SSO2368, PAE2420, TK1449, 1491, TVN0174, PH1093, | |
| | VF2267, Saci_0971, VV11364, CMT389C, VV3008 | |
| Aspartate kinase | Cgl0251, NCgl0247, CE0220, DIP0277, jk1998, nfa3180, | C. glutamicum and |
| (ask) | Mb3736c, MT3812, Rv3709c, ML2323, MAP0311c, Tfu_0043, | others |
| | Francci3_0262, SCO3615, SAV4559, Lxx03450, PPA2148, | |
| | CHY_1909, MCA0390, cbdb_A1731, TWT708, TW725, | |
| | Gmet_1880, DET1633, GSU1799, Moth_1304, Tcr_1589, | |
| | Mfla_0567, HCH_05208, PSPPH_3511, Psyr_3555, | |
| | PSPTO1843, CV1018, STH1686, NMA1701, Tbd_0969, | |
| | NMB1498, Pcar_1006, Daro_2515, Csal_0626, Tmden_1650, | |
| | PA0904, PP4473, Sde_1300, HH0618, NGO0956, | |
| | ACIAD1252, PFL_4505, ebA637, Noc_0927, WS1729, | |
| | Pcryo_1639, Psyc_1461, Pfl_4274, LIC12909, LA0693, | |
| | Rru_A0743, NE2132, RB8926, Cj0582, Nmul_A1941, | |
| | SYN_02781, TTHA0534, CJE0685, BURPS1710b_2677, | |
| | BPSL2239, BMA1652, RSc1171, TTC0166, RPA0604, | |
| | BTH_I1945, Bpro_2860, Rmet_1089, Reut_A1126, RPD_0099, | |
| | Bxe_A1630, Bcep18194_A5380, aq_1152, RPB_0077, | |
| | Rfer_1353, RPC_0514, BH3096, BLi02996, BL00324, | |
| | amb1612, tlr1833, jhp1150, blr0216, Dde_2048, BB1739, | |
| | BPP2287, BP1913, DVU1913, Nwi_0379, ZMO1653, | |
| | Jann_3191, HP1229, Saro_3304, Nham_0472, CBU_1051, | |
| | slr0657, SPO3035, Synpcc7942_1001, BG10350, | |
| | BruAb1_1850, BAB1_1874, BMEI0189, BT9727_1658, | |
| | syc0544_d, BR1871, gll1774, BC1748, mll3437, BCE1883, | |
| | ELI_14545, RSP_1849, BCZK1623, BAS1676, BA_2315, | |
| | GBAA1811, BA1811, Ava_3642, alr3644, PSHAa0533, | |
| | AGR_L_1357, Atu4172, lin1198, BH04030, PMT9312_1740, | |
| | SMc02438, CYA_1747, RHE_CH03758, lmo1235, | |
| | LMOf2365_1244, PMN2A_1246, CC0843, Pro1808, BQ03060, | |
| | PMT0073, Syncc9902_0068, GOX0037, CYB_0217 | |
| Homoserine | Cgl0652, CE0678, CE0678, cg0754, DIP0623, jk1695, nfa9220, RHA1_ro06236, | C. glutamicum and |
| Succinyltransferase | MAP3458, MAV_4316, MSMEG_1651, Mmcs_1207, | others |
| (metA) | ML0682, Mb3373, Rv3341, MT3444, Tfu_2822, Arth_1318, Francci3_2831, | |
| | Lxx18950, FRAAL4363, Cag_1206, Adeh_1400, Plut_0593, | |
| | CT0605, CHY_1903, Moth_1308, Ava_4076, STH1685, SRU_0480, | |
| | Mbur_0798, Mhun_2201, RPC_4281 Msp_0676 | |
| homoserine | Cgl1183, CE1289, cg1337, DIP1036, jk1352, nfa10490, RHA1_ro01488, | C. glutamicum and |
| dehydrogenase | MSMEG_4957, Mmcs_3896, MAV_1509, Mb1326, Rv1294, | others |
| (hom) | MT1333, MAP2468c, ML1129, SAV2918, SCO5354, FRAAL5951, | |
| | Francci3_3725, Tfu_2424, Acel_0630 | |
| Homoserine kinase | Cgl1184, cg0307, CE0221, DIP0279, jk1997, RHA1_ro04292, nfa3190, | C. glutamicum and |
| (hsk) | Mmcs_4888, MSMEG_6256, MAP0310c, MAV_0394, Mb3735c, | others |
| | MT3811, Rv3708c, Acel_2011, ML2322, PPA0318, Lxx03460, | |
| | SCO2640, SAV5397, CC3485 | |
| D-methionine | YP_224930, NP_599871, NP_737241, NP_938985, NP_938984, YP_701727, | C. glutamicum and |
| binding lipoprotein | YP_251505, YP_120623, YP_062481, YP_056445, ZP_00121548, | others |
| (metQ) | NP_696133, YP_034633, YP_034633, YP_081895, ZP_00390696, | |
| | YP_016928, YP_026579, NP_842863, YP_081895, | |
| | ZP_00240243, NP_976671 | |
| mcbR | cg3253, CE2788, DIP2274, jk0101, nfa21280, MSMEG_4517Lxx16190, | C. glutamicum and |
| | SCO4454, Bcep18194_A3587, Bamb_0404, Bcen2424_0499, | others |
| | Bcen_2606, Ava_4037, BTH_I2940, RHA1_ro02712, BMA10299_A1735, | |
| | BMASAVP1_A0031, BMA2807, BURPS1710b_3614 | |

The above accession numbers are the official accession numbers of Genbank or are synonyms for accession numbers which have cross-references at Genbank. These numbers can be searched and found at http://www.ncbi.nlm.nih.gov/.

A general overview is given below on how to increase and decrease the amount and/or activity of polypeptides and genes in C. glutamicum. The skilled person can rely on this information when putting embodiments besides those disclosed in the examples below into practice.

Increasing or Introducing the Amount and/or Activity

With respect to increasing the amount, two basic scenarios can be differentiated. In the first scenario, the amount of the enzyme is increased by expression of an exogenous version of the respective protein. In the other scenario, expression of the endogenous protein is increased by influencing the activity of e.g. the promoter and/or enhancers ribosomal binding sites element and/or other regulatory activities that regulate the activities of the respective proteins either on a transcriptional, translational or post-translational level.

Thus, the increase of the activity and the amount of a protein may be achieved via different routes, e.g. by switching off inhibitory regulatory mechanisms at the transcriptional, translational, and protein level or by increase of gene expression of a nucleic acid coding for these proteins in comparison with the starting organism, e.g. by inducing endogenous transketolase by a strong promoter and/or by introducing nucleic acids encoding for transketolase.

In one embodiment, the increase of the amount and/or activity of the enzymes of Table 1 or Table 2 is achieved by introducing nucleic acids encoding the enzymes of Table 1 or Table 2 into the Coryneform bacteria, preferably C. glutamicum.

In principle, every protein of different organisms with an enzymatic activity of the proteins listed in Table 1 or 2, can be used. With genomic nucleic acid sequences of such enzymes from eukaryotic sources containing introns, already processed nucleic acid sequences like the corresponding cDNAs are to be used in the case as the host organism is not capable or cannot be made capable of splicing the corresponding mRNAs. All nucleic acids mentioned in the description can be, e.g., an RNA, DNA or cDNA sequence.

According to the present invention, increasing or introducing the amount of a protein typically comprises the following steps:

a) production of a vector comprising the following nucleic acid sequences, preferably DNA sequences, in 5′-3′-orientation:
a promoter sequence functional in the organisms of the invention
operatively linked thereto a DNA sequence coding for a protein of e.g. Table 1, functional homologues, functional fragments or functional mutated versions thereof
a termination sequence functional in the organisms of the invention
   b) transfer of the vector from step a) to the organisms of the invention such as C. glutamicum and, optionally, integration into the respective genomes.

As set out above, functional fragments relate to fragments of nucleic acid sequences coding for enzymes of e.g. Table 1 or 2, the expression of which still leads to proteins having the enzymatic activity of the respective full length protein.

The above-mentioned method can be used for increasing the expression of DNA sequences coding for enzymes of e.g. Table 1 or functional fragments thereof. The use of such vectors comprising regulatory sequences, like promoter and termination sequences are, is known to the person skilled in the art. Furthermore, the person skilled in the art knows how a vector from step a) can be transferred to organisms such as C. glutamicum and which properties a vector must have to be able to be integrated into their genomes.

According to the present invention, an increase of the gene expression of a nucleic acid encoding an enzyme of Table 1 or 2 is also understood to be the manipulation of the expression of the endogenous respective endogenous enzymes of an organism, in particular of C. glutamicum. This can be achieved, e.g., by altering the promoter DNA sequence for genes encoding these enzymes. Such an alteration, which causes an altered, preferably increased, expression rate of these enzymes can be achieved by replacement wit strong promoters and by deletion and/or insertion of DNA sequences.

An alteration of the promoter sequence of endogenous genes usually causes an alteration of the expressed amount of the gene and therefore also an alteration of the activity detectable in the cell or in the organism.

Furthermore, an altered and increased expression, respectively, of an endogenous gene can be achieved by a regulatory protein, which does not occur in the transformed organism, and which interacts with the promoter of these genes. Such a regulator can be a chimeric protein consisting of a DNA binding domain and a transcription activator domain, as e.g. described in WO 96/06166.

A further possibility for increasing the activity and the content of endogenous genes is to up-regulate transcription factors involved in the transcription of the endogenous genes, e.g. by means of overexpression. The measures for overexpression of transcription factors are known to the person skilled in the art.

The expression of endogenous enzymes such as those of Table 1 can e.g. be regulated via the expression of aptamers specifically binding to the promoter sequences of the genes. Depending on the aptamer binding to stimulating or repressing promoter regions, the amount of the enzymes of Table 2 can e.g. be increased.

Furthermore, an alteration of the activity of endogenous genes can be achieved by targeted mutagenesis of the endogenous gene copies.

An alteration of the endogenous genes coding for the enzymes of e.g. Table 1 can also be achieved by influencing the post-translational modifications of the enzymes. This can happen e.g. by regulating the activity of enzymes like kinases or phosphatases involved in the post-translational modification of the enzymes by means of corresponding measures like overexpression or gene silencing.

In another embodiment, an enzyme may be improved in efficiency, or its allosteric control region destroyed such that feedback inhibition of production of the compound is prevented. Similarly, a degradative enzyme may be deleted or modified by substitution, deletion, or addition such that its degradative activity is lessened for the desired enzyme of Table 1 without impairing the viability of the cell. In each case, the overall yield, rate of production or amount of methionine be increased.

It is also possible that such alterations in the proteins of e.g. Table 1 may improve the production of other fine chemicals such as other sulfur containing compounds like cysteine or glutathione, other amino acids, vitamins, cofactors, nutraceuticals, nucleic acids, nucleosides, and trehalose. Metabolism of any one compound can be intertwined with other biosynthetic and degradative pathways within the cell, and necessary cofactors, intermediates, or substrates in one pathway may be supplied or limited by another such pathway. Therefore, by modulating the activity of one or more of the proteins of Table 1, the amount, efficiency and rate of other fine chemicals besides methionine may be positively impacted.

These aforementioned strategies for increasing or introducing the amount and/or activity of the enzymes of Table 1 are not meant to be limiting; variations on these strategies will be readily apparent to one of ordinary skill in the art.

Reducing the Amount and/or Activity of Enzymes

It has been set out above that it may be preferred to use starting organism which have already been optimized for methionine production. In C. glutamicum one may, for example, downregulate the activity of metQ.

For reducing the amount and/or activity of enzymes, various strategies are available.

The expression of endogenous enzymes such as those of Table 2 can e.g. be regulated via the expression of aptamers specifically binding to the promoter sequences of the genes. Depending on the aptamer binding to stimulating or repressing promoter regions, the amount and thus, in this case, the activity of the enzymes of Table 2 can e.g. be reduced.

Aptamers can also be designed in a way as to specifically bind to the enzymes themselves and to reduce the activity of the enzymes by e.g. binding to the catalytic center of the respective enzymes. The expression of aptamers is usually achieved by vector-based overexpression (see above) and is, as well as the design and the selection of aptamers, well known to the person skilled in the art (Famulok et al., (1999) Curr Top Microbiol Immunol., 243, 123-36).

Furthermore, a decrease of the amount and the activity of the endogenous enzymes of Table 2 can be achieved by means of various experimental measures, which are well known to the person skilled in the art. These measures are usually summarized under the term “gene silencing”. For example, the expression of an endogenous gene can be silenced by transferring an above-mentioned vector, which has a DNA sequence coding for the enzyme or parts thereof in antisense order, to organisms such as C. glutamicum. This is based on the fact that the transcription of such a vector in the cell leads to an RNA, which can hybridize with the mRNA transcribed by the endogenous gene and therefore prevents its translation.

In principle, the antisense strategy can be coupled with a ribozyme method. Ribozymes are catalytically active RNA sequences, which, if coupled to the antisense sequences, cleave the target sequences catalytically (Tanner et al., (1999) FEMS Microbiol Rev. 23 (3), 257-75). This can enhance the efficiency of an antisense strategy.

To create a homologous recombinant microorganism, a vector is prepared which contains at least a portion of gene coding for an enzyme of Table 1 into which a deletion, addition or substitution has been introduced to thereby alter, e.g., functionally disrupt, the endogenous gene.

In one embodiment, the vector is designed such that, upon homologous recombination, the endogenous gene is functionally disrupted (i.e., no longer encodes a functional protein). Alternatively, the vector can be designed such that, upon homologous recombination, the endogenous gene is mutated or otherwise altered but still encodes functional protein, e.g., the upstream regulatory region can be altered to thereby alter the expression of the endogenous enzymes of e.g. Table 2. This approach can have the advantage that expression of an enzyme is not completely abolished, but reduced to the required minimum level. The skilled person knows which vectors can be used to replace or delete endogenous sequences. For. C. glutamicum, such vectors include pK19 and pCLIK int sacB. A specific description for disrupting chromosomal sequences in C. glutamicum is provided below.

Furthermore, gene repression is possible by reducing the amount of transcription factors.

Factors inhibiting the target protein itself can also be introduced into a cell. The protein-binding factors may e.g. be the above-mentioned aptamers (Famulok et al., (1999) Curr Top Microbiol Immunol. 243, 123-36).

As further protein-binding factors, the expression of which can cause a reduction of the amount and/or the activity of the enzymes of table 1, enzyme-specific antibodies may be considered. The production of recombinant enzyme-specific antibodies such as single chain antibodies is known in the art. The expression of antibodies is also known from the literature (Fiedler et al., (1997) Immunotechnology 3, 205-216; Maynard and Georgiou (2000) Annu. Rev. Biomed. Eng. 2, 339-76).

The mentioned techniques are well known to the person skilled in the art. Therefore, the skilled also knows the typical size that a nucleic acid constructs used for e.g. antisense methods must have and which complementarity, homology or identity, the respective nucleic acid sequences must have. The terms complementarity, homology, and identity are known to the person skilled in the art.

The term complementarity describes the capability of a nucleic acid molecule to hybridize with another nucleic acid molecule due to hydrogen bonds between two complementary bases. The person skilled in the art knows that two nucleic acid molecules do not have to display a complementarity of 100% in order to be able to hybridize with each other. A nucleic acid sequence, which is to hybridize with another nucleic acid sequence, is preferably at least 30%, at least 40%, at least 50%, at least 60%, preferably at least 70%, particularly preferred at least 80%, also particularly preferred at least 90%, in particular preferred at least 95% and most preferably at least 98 or 100%, respectively, complementary with said other nucleic acid sequence.

The hybridization of an antisense sequence with an endogenous mRNA sequence typically occurs in vivo under cellular conditions or in vitro. According to the present invention, hybridization is carried out in vivo or in vitro under conditions that are stringent enough to ensure a specific hybridization.

Stringent in vitro hybridization conditions are known to the person skilled in the art and can be taken from the literature (see e.g. Sambrook et al., Molecular Cloning, Cold Spring Harbor Press (2001)). The term “specific hybridization” refers to the case wherein a molecule preferentially binds to a certain nucleic acid sequence under stringent conditions, if this nucleic acid sequence is part of a complex mixture of e.g. DNA or RNA molecules.

The term “stringent conditions” therefore refers to conditions, under which a nucleic acid sequence preferentially binds to a target sequence, but not, or at least to a significantly reduced extent, to other sequences.

Stringent conditions are dependent on the circumstances. Longer sequences specifically hybridize at higher temperatures. In general, stringent conditions are chosen in such a way that the hybridization temperature lies about 5° C. below the melting point (Tm) of the specific sequence with a defined ionic strength and a defined pH value. Tm is the temperature (with a defined pH value, a defined ionic strength and a defined nucleic acid concentration), at which 50% of the molecules, which are complementary to a target sequence, hybridize with said target sequence. Typically, stringent conditions comprise salt concentrations between 0.01 and 1.0 M sodium ions (or ions of another salt) and a pH value between 7.0 and 8.3. The temperature is at least 30° C. for short molecules (e.g. for such molecules comprising between 10 and 50 nucleic acids). In addition, stringent conditions can comprise the addition of destabilizing agents like e.g. form amide. Typical hybridization and washing buffers are of the following composition.

### Pre-Hybridization Solution:

0.5% SDS
5×SSC
50 mM NaPO₄, pH 6.8
0.1% Na-pyrophosphate
5×Denhardt's reagent
100 μg/salmon sperm
   Hybridization solution:
Pre-hybridization solution
1×10⁶ cpm/ml probe (5-10 min 95° C.)

### 20×SSC:

3 M NaCl
0.3 M sodium citrate
ad pH 7 with HC₁₋₅₀
   50×Denhardt's reagent:
5 g Ficoll
5 g polyvinylpyrrolidone
5 g Bovine Serum Albumin
ad 500 ml A. dest.

A typical procedure for the hybridization is as follows:

Optional: wash Blot 30 min in 1×SSC/0.1% SDS at 65° C.
Pre-hybridization: at least 2 h at 50-55° C.
Hybridization: over night at 55-60° C.

| | | | |
|---|---|---|---|
| Washing: | 05 min | 2x SSC/0.1% SDS | Hybridization temperature |
| | 30 min | 2x SSC/0.1% SDS | Hybridization temperature |
| | 30 min | 1x SSC/0.1% SDS | Hybridization temperature |
| | 45 min | 0.2x SSC/0.1% SDS | 65° C. |
| | 5 min | 0.1x SSC | room temperature |

For antisense purposes complementarity over sequence lengths of 100 nucleic acids, 80 nucleic acids, 60 nucleic acids, 40 nucleic acids and 20 nucleic acids may suffice. Longer nucleic acid lengths will certainly also suffice. A combined application of the above-mentioned methods is also conceivable.

If, according to the present invention, DNA sequences are used, which are operatively linked in 5′-3′-orientation to a promoter active in the organism, vectors can, in general, be constructed, which, after the transfer to the organism's cells, allow the overexpression of the coding sequence or cause the suppression or competition and blockage of endogenous nucleic acid sequences and the proteins expressed there from, respectively.

The activity of a particular enzyme may also be reduced by over-expressing a non-functional mutant thereof in the organism. Thus, a non-functional mutant which is not able to catalyze the reaction in question, but that is able to bind e.g. the substrate or co-factor, can, by way of over-expression out-compete the endogenous enzyme and therefore inhibit the reaction. Further methods in order to reduce the amount and/or activity of an enzyme in a host cell are well known to the person skilled in the art.

According to the present invention, non-functional enzymes have essentially the same nucleic acid sequences and amino acid sequences, respectively, as functional enzymes and functionally fragments thereof, but have, at some positions, point mutations, insertions or deletions of nucleic acids or amino acids, which have the effect that the non-functional enzyme are not, or only to a very limited extent, capable of catalyzing the respective reaction. These non-functional enzymes may not be intermixed with enzymes that still are capable of catalyzing the respective reaction, but which are not feedback regulated anymore. According to the present invention, the term “non-functional enzyme” does not comprise such proteins having no substantial sequence homology to the respective functional enzymes at the amino acid level and nucleic acid level, respectively. Proteins unable to catalyse the respective reactions and having no substantial sequence homology with the respective enzyme are therefore, by definition, not meant by the term “non-functional enzyme” of the present invention. Non-functional enzymes are, within the scope of the present invention, also referred to as inactivated or inactive enzymes.

Therefore, non-functional enzymes of e.g. Table 2 according to the present invention bearing the above-mentioned point mutations, insertions, and/or deletions are characterized by an substantial sequence homology to the wild type enzymes of e.g. Table 2 according to the present invention or functionally equivalent parts thereof. For determining a substantial sequence homo logy, the above describded identity grades are to applied.

### Vectors and Host Cells

One aspect of the invention pertains to vectors, preferably expression vectors, containing a nucleic acid sequences as mentioned above. As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include other forms of expression vectors, such as viral vectors, which serve equivalent functions.

The recombinant expression vectors of the invention may comprise a nucleic acid as mentioned above in a form suitable for expression of the respective nucleic acid in a host cell, which means that the recombinant expression vectors include one or more regulatory sequences, selected on the basis of the host cells to be used for expression, which are operatively linked to the nucleic acid sequence to be expressed.

For the purposes of the present invention, an operative link is understood to be the sequential arrangement of promoter, coding sequence, terminator and, optionally, further regulatory elements in such a way that each of the regulatory elements can fulfill its function, according to its determination, when expressing the coding sequence.

Within a recombinant expression vector, “operably linked” is thus intended to mean that the nucleic acid sequence of interest is linked to the regulatory sequence (s) in a manner which allows for expression of the nucleic acid sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleic acid sequence in many types of host cell and those which direct expression of the nucleic acid sequence only in certain host cells. Preferred regulatory sequences are, for example, promoters such as cos-, tac-, trp-, tet-, trp-, tet-, lpp-, lac-, lpp-lac-, lacIq-, T7-, T5-, T3-, gal-, trc-, ara-, SP6-, arny, SP02, SOD, EFTu, EFTs, GroEL, MetZ (all from C. glutamicum), which are used preferably in bacteria. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by the above-mentioned nucleic acid sequences.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve three 4 purposes: 1) to increase expression of recombinant protein; 2) to increase the solubility of the recombinant protein; and 3) to aid in the purification of the recombinant protein by acting as a ligand in affinity purification 4) to provide a “tag” for later detection of the protein. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively.

Examples of suitable inducible non-fusion expression vectors for Coryneform bacteria include pHM1519, pBL1, pSA77 or pAJ667 (Pouwels et al., eds. (1985) Cloning Vectors. Elsevier: New York IBSN 0 444 904018). Examples of suitable C. glutamicum and E coli shuttle vectors are e.g. pK19, pClik5aMCS pCLIKint sacB or can be found in Eikmanns et al (Gene. (1991) 102, 93-8) and in the following publications and patent applications (Schafer A, et al. J. Bacteriol. 1994 176: 7309-7319, Bott, M. and Eggeling, L., eds. Handbook of Corynebacterium glutamicum. CRC Press LLC, Boca Raton, Fla. WO2006069711, WO2006069711). For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

Vector DNA can be introduced into prokaryotic via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 3rd ed., Cold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, kanamycine, tetracycline, chloramphenicol, ampicillin and methotrexate. Nucleic acid encoding a selectable marker can be introduced into a host cell on the same vector as that encoding the above-mentioned modified nucleic acid sequences or can be introduced on a separate vector. Cells stably transfected with the introduced nucleic acid can be identified by drug selection (e.g., cells that have incorporated the selectable marker gene will survive, while the other cells die).

In another embodiment, recombinant microorganisms can be produced which contain selected systems which allow for regulated expression of the introduced gene. For example, inclusion of one of the above-mentioned nucleic acid sequences on a vector placing it under control of the lac operon permits expression of the gene only in the presence of IPTG. Such regulatory systems are well known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Growth of C. glutamicum-Media and Culture Conditions

A general teaching will be given below as to the cultivation of C.glutamicum. Adaptions will be obvious to the skilled person Corresponding information may be retrieved from standard textbooks for cultivation of E. coli.

Genetically modified Corynebacteria are typically cultured in synthetic or natural growth media. A number of different growth media for Corynebacteria are both well and readily available (Lieb et al. (1989) Appl. Microbiol. Biotechnol., 32: 205-210; von der Osten et al. (1998) Biotechnology Letters, 11: 11-16; Patent DE 4,120,867; Lieb1 (1992) “The Genus Corynebacterium, in: The Procaryotes, Volume II, Balows, A. et al., eds. Springer-Verlag).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂S0₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 minutes at 1.5 bar and 121° C.) or by sterile filtration. The components can either be sterilized together or, if necessary, separately.

All media components may be present at the beginning of growth, or they can optionally be added continuously or batch wise. Culture conditions are defined separately for each experiment.

The temperature should be in a range between 15° C. and 45° C. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄ OH during growth. If complex medium components such as yeast extract are utilized, the necessity for additional buffers may be reduced, due to the fact that many complex compounds have high buffer capacities. If a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml or 250 ml shake flasks are used, filled with 10% (by volume) of the required growth medium. The flasks should be shaken on a rotary shaker (amplitude 25 mm) using a speed-range of 100-300′ rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD600 of 0.5-1.5 using cells grown on agar plates, such as CM plates (10 g/1 glucose, 2.5 g/1 NaCl, 2 g/1 urea, 10 g/1 polypeptone, 5 g/1 yeast extract, 5 g/1 meat extract, 2 g/1 urea, 10 g/1 polypeptone, 5 g/1 yeast extract, 5 g/1 meat extract, 22 g/1 agar, pH 6.8 with 2M NaOH) that had been incubated at 30 C. Inoculation of the media is accomplished by either introduction of a saline suspension of C. glutamicum cells from CM plates or addition of a liquid preculture of this bacterium. Other incubation methods can be taken from WO2007012078.

### General Methods

Protocols for general methods can be found in Handbook on Corynebacterium glutamicum, (2005) eds.: L. Eggeling, M. Bott., Boca Raton, CRC Press, at Martin et al. (Biotechnology (1987) 5, 137-146), Guerrero et al. (Gene (1994), 138, 35-41), Tsuchiya und Morinaga (Biotechnology (1988), 6, 428-430), Eikmanns et al. (Gene (1991), 102, 93-98), EP 0 472 869, U.S. Pat. No. 4,601,893, Schwarzer and Piihler (Biotechnology (1991), 9, 84-87, Reinscheid et al. (Applied and Environmental Microbiology (1994), 60, 126-132), LaBarre et al. (Journal of Bacteriology (1993), 175, 1001-1007), WO 96/15246, Malumbres et al. (Gene (1993), 134, 15-24), in JP-A-10-229891, at Jensen und Hammer (Biotechnology and Bioengineering (1998), 58, 191-195), Makrides (Microbiological Reviews (1996), 60, 512-538) in WO2006069711, in WO2007012078 and in well known textbooks of genetic and molecular biology.

### Strains, Media and Plasmids

Strains can be taken e.g. from the following list:

Corynebacterium glutamicum ATCC 13032,
Corynebacterium acetoglutamicum ATCC 15806,
Corynebacterium acetoacidophilum ATCC 13870,
Corynebacterium thermoaminogenes PERM BP-1539,
Corynebacterium melassecola ATCC 17965,
Brevibacterium flavum ATCC 14067,
Brevibacterium lactofermentum ATCC 13869, and
Brevibacterium divaricatum ATCC 14020 or strains which have been derived therefrom such as Corynebacterium glutamicum KFCC10065, DSM 17322 or
Corynebacterium glutamicum ATCC21608

### Recombinant DNA Technology

Protocols can be found in: Sambrook, J., Fritsch, E. F., and Maniatis, T., in Molecular Cloning: A Laboratory Manual, 3^{rd} edition (2001) Cold Spring Harbor Laboratory Press, NY, Vol. 1, 2, 3, and Handbook on Corynebacterium glutamicum (2005) eds. L. Eggeling, M. Bott., Boca Raton, CRC Press.

### Quantification of Amino Acids and Methionine Intermediates.

The analysis is done by HPLC (Agilent 1100, Agilent, Waldbronn, Germany) with a guard cartridge and a Synergi 4 μm column (MAX-RP 80 Å, 150*4.6 mm) (Phenomenex, Aschaffenburg, Germany). Prior to injection the analytes are derivatized using o-phthaldialdehyde (OPA) and mercaptoethanol as reducing agent (2-MCE). Additionally sulfhydryl groups are blocked with iodoacetic acid. Separation is carried out at a flow rate of 1 ml/min using 40 mM NaH₂PO₄ (eluent A, pH=7.8, adjusted with NaOH) as polar and a methanol water mixture (100/1) as non-polar phase (eluent B). The following gradient is applied: Start 0% B; 39 min 39% B; 70 min 64% B; 100% B for 3.5 min; 2 min 0% B for equilibration. Derivatization at room temperature is automated as described below. Initially 0.5 μl of 0.5% 2-MCE in bicine (0.5M, pH 8.5) are mixed with 0.5 μl cell extract. Subsequently 1.5 μl of 50 mg/ml iodoacetic acid in bicine (0.5M, pH 8.5) are added, followed by addition of 2.5 μl bicine buffer (0.5M, pH 8.5). Derivatization is done by adding 0.5 μl of 10 mg/ml OPA reagent dissolved in 1/45/54 v/v/v of 2-MCE/MeOH/bicine (0.5M, pH 8.5). Finally the mixture is diluted with 32 μl H₂O. Between each of the above pipetting steps there is a waiting time of 1 min. A total volume of 37.5 μl is then injected onto the column. Note, that the analytical results can be significantly improved, if the auto sampler needle is periodically cleaned during (e.g. within waiting time) and after sample preparation. Detection is performed by a fluorescence detector (340 nm excitation, emission 450 nm, Agilent, Waldbronn, Germany). For quantification α-amino butyric acid (ABA) was is as internal standard

### Definition of Recombination Protocol

In the following it will be described how a strain of C. glutamicum with increased efficiency of methionine production can be constructed implementing the findings of the above predictions. Before the construction of the strain is described, a definition of a recombination event/protocol is given that will be used in the following.

“Campbell in,” as used herein, refers to a transformant of an original host cell in which an entire circular double stranded DNA molecule (for example a plasmid being based on pCLIK int sacB or pK19 has integrated into a chromosome by a single homologous recombination event (a cross-in event), and that effectively results in the insertion of a linearized version of said circular DNA molecule into a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” cell can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to be approximately the same length, preferably with a difference of less than 200 base pairs and most preferably with the shorter of the two being at least 70% of the length of the longer in base pairs. A description of the Campbell in and out method can be taken from WO2007012078.

### EXAMPLES

The following experiments demonstrate how overexpression of C. glutamicum transketolase leads to increased methionine production. These examples are however in no way meant to limit the invention in any way.

### Shake Flask Experiments and HPLC Assay

Shake flasks experiments, with the standard Molasses Medium, were performed with strains in duplicate or quadruplicate. Molasses Medium contained in one liter of medium: 40 g glucose; 60 g molasses; 20 g (NH₄)₂ SO₄; 0.4 g MgSO₄*7H₂O; 0.6 g KH₂PO₄; 10 g yeast extract (DIFCO); 5 ml of 400 mM threonine; 2 mgFeSO₄.7H₂O; 2 mg of MnSO₄.H₂O; and 50 g CaCO₃ (Riedel-de Haen), with the volume made up with ddH₂O. The pH was adjusted to 7.8 with 20% NH₄OH, 20 ml of continuously stirred medium (in order to keep CaCO₃ suspended) was added to 250 ml baffled Bellco shake flasks and the flasks were autoclaved for 20 min. Subsequent to autoclaving, 4 ml of “4B solution” was added per liter of the base medium (or 80 μl/flask). The “4B solution” contained per liter: 0.25 g of thiamine hydrochloride (vitamin B1), 50 mg of cyanocobalamin (vitamin B12), 25 mg biotin, 1.25 g pyridoxine hydrochloride (vitamin B6) and was buffered with 12.5 mM KPO₄, pH 7.0 to dissolve the biotin, and was filter sterilized. Cultures were grown in baffled flasks covered with Bioshield paper secured by rubber bands for 48 hours at 28° C. or 30° C. and at 200 or 300 rpm in a New Brunswick Scientific floor shaker. Samples were taken at 24 hours and/or 48 hours. Cells were removed by centrifugation followed by dilution of the supernatant with an equal volume of 60% acetonitrile and then membrane filtration of the solution using Centricon 0.45 μm spin columns. The filtrates were assayed using HPLC for the concentrations of methionine, glycine plus homoserine, O-acetylhomoserine, threonine, isoleucine, lysine, and other indicated amino acids.

For the HPLC assay, filtered supernatants were diluted 1:100 with 0.45 μm filtered 1 mM Na₂EDTA and 1 μl of the solution was derivatized with OPA reagent (AGILENT) in Borate buffer (80 mM NaBO₃, 2.5 mM EDTA, pH 10.2) and injected onto a 200×4.1 mm Hypersil 5μ AA-ODS column run on an Agilent 1100 series HPLC equipped with a G1321A fluorescence detector (AGILENT). The excitation wavelength was 338 nm and the monitored emission wavelength was 425 nm. Amino acid standard solutions were chromatographed and used to determine the retention times and standard peak areas for the various amino acids. Chem Station, the accompanying software package provided by Agilent, was used for instrument control, data acquisition and data manipulation. The hardware was an HP Pentium 4 computer that supports Microsoft Windows NT 4.0 updated with a Microsoft Service Pack (SP6a).

### Experiment 1—Generation of the M2014 Strain

C. glutamicum strain ATCC 13032 was transformed with DNA A (also referred to as pH273) (SEQ ID NO: 24) and “Campbelled in” to yield a “Campbell in” strain. The “Campbell in” strain was then “Campbelled out” to yield a “Campbell out” strain, M440, which contains a gene encoding a feedback resistant homoserine dehydrogenase enzyme (hom^{fbr}). The resultant homoserine dehydrogenase protein included an amino acid change where S393 was changed to F393 (referred to as Hsdh S393F).

The strain M440 was subsequently transformed with DNA B (also referred to as pH373) (SEQ ID NO: 25) to yield a “Campbell in” strain. The “Campbell in” strain were then “Campbelled out” to yield a “Campbell out” strain, M603, which contains a gene encoding a feedback resistant aspartate kinase enzyme (Ask^{fbr}) (encoded by lysC). In the resulting aspartate kinase protein, T311 was changed to I311 (referred to as LysC T311I).

It was found that the strain M603 produced about 17.4 mM lysine, while the ATCC13032 strain produced no measurable amount of lysine. Additionally, the M603 strain produced about 0.5 mM homoserine, compared to no measurable amount produced by the ATCC13032 strain, as summarized in Table 3.

TABLE 3
Amounts of homoserine, O-acetylhomoserine, methionine and lysine
produced by strains ATCC13032 and M603

| | | O-acetyl | | |
|---|---|---|---|---|
| | Homoserine | homoserine | Methionine | Lysine |
| Strain | (mM) | (mM) | (mM) | (mM) |
| ATCC13032 | 0.0 | 0.4 | 0.0 | 0.0 |
| M603 | 0.5 | 0.7 | 0.0 | 17.4 |

The strain M603 was transformed with DNA C (also referred to as pH304) (SEQ ID NO:26) to yield a “Campbell in” strain, which was then “Campbelled out” to yield a “Campbell out” strain, M690. The M690 strain contained a PgroES promoter upstream of the metH gene (referred to as P₄₉₇ metH). The sequence of the P₄₉₇ promoter is depicted in SEQ ID NO: 21. The M690 strain produced about 77.2 mM lysine and about 41.6 mM homoserine, as shown below in Table 4.

TABLE 4
Amounts of homoserine, O-acetyl homoserine, methionine and lysine
produced by the strains M603 and M690

| | | O-acetyl | | |
|---|---|---|---|---|
| | Homoserine | homoserine | Methionine | Lysine |
| Strain | (mM) | (mM) | (mM) | (mM) |
| M603 | 0.5 | 0.7 | 0.0 | 17.4 |
| M690 | 41.6 | 0.0 | 0.0 | 77.2 |

The M690 strain was subsequently mutagenized as follows: an overnight culture of M603, grown in BHI medium (BECTON DICKINSON), was washed in 50 mM citrate buffer pH 5.5, treated for 20 min at 30° C. with N-methyl-N-nitrosoguanidine (10 mg/ml in 50 mM citrate pH 5.5). After treatment, the cells were again washed in 50 mM citrate buffer pH 5.5 and plated on a medium containing the following ingredients: (all mentioned amounts are calculated for 500 ml medium) 10 g (NH₄)₂SO₄; 0.5 g KH₂PO₄; 0.5 g K₂HPO₄; 0.125 g MgSO₄*7H₂O; 21 g MOPS; 50 mg CaCl₂; 15 mg protocatechuic acid; 0.5 mg biotin; 1 mg thiamine; and 5 g/l D,L-ethionine (SIGMA CHEMICALS, CATALOG #E5139), adjusted to pH 7.0 with KOH. In addition the medium contained 0.5 ml of a trace metal solution composed of: 10 g/l FeSO₄*7H₂O; 1 g/l MnSO₄*H₂O; 0.1 g/l ZnSO₄*7H₂O; 0.02 g/l CuSO₄; and 0.002 g/l NiCl₂*6H₂O, all dissolved in 0.1 M HCl. The final medium was sterilized by filtration and to the medium, 40 mls of sterile 50% glucose solution (40 ml) and sterile agar to a final concentration of 1.5% were added. The final agar containing medium was poured to agar plates and was labeled as minimal-ethionine medium. The mutagenized strains were spread on the plates (minimal-ethionine) and incubated for 3-7 days at 30° C. Clones that grew on the medium were isolated and restreaked on the same minimal-ethionine medium. Several clones were selected for methionine production analysis.

Methionine production was analyzed as follows. Strains were grown on CM-agar medium for two days at 30° C., which contained: 10 g/l D-glucose, 2.5 g/l NaCl; 2 g/l urea; 10 g/l Bacto Peptone (DIFCO); 5 g/l Yeast Extract (DIFCO); 5 g/l Beef Extract (DIFCO); 22 g/l Agar (DIFCO); and which was autoclaved for 20 min at about 121° C.

After the strains were grown, cells were scraped off and resuspended in 0.15 M NaCl. For the main culture, a suspension of scraped cells was added at a starting OD of 600 nm to about 1.5 to 10 ml of Medium II (see below) together with 0.5 g solid and autoclaved CaCO₃ (RIEDEL DE HAEN) and the cells were incubated in a 100 ml shake flask without baffles for 72 h on a orbital shaking platform at about 200 rpm at 30° C. Medium II contained: 40 g/l sucrose; 60 g/l total sugar from molasses (calculated for the sugar content); 10 g/l (NH₄)₂SO₄; 0.4 g/l MgSO₄*7H₂O; 0.6 g/l KH₂PO₄; 0.3 mg/l thiamine*HCl; 1 mg/l biotin; 2 mg/l FeSO₄; and 2 mg/l MnSO₄. The medium was adjusted to pH 7.8 with NH₄OH and autoclaved at about 121° C. for about 20 min). After autoclaving and cooling, vitamin B₁₂ (cyanocobalamine) (SIGMA CHEMICALS) was added from a filter sterile stock solution (200 μg/ml) to a final concentration of 100 μg/l.

Samples were taken from the medium and assayed for amino acid content. Amino acids produced, including methionine, were determined using the Agilent amino acid method on an Agilent 1100 Series LC System HPLC. (AGILENT). A pre-column derivatization of the sample with ortho-pthalaldehyde allowed the quantification of produced amino acids after separation on a Hypersil AA-column (AGILENT).

Clones that showed a methionine titer that was at least twice that in M690 were isolated. One such clone, used in further experiments, was named M1197 and was deposited on May 18, 2005, at the DSMZ strain collection as strain number DSM 17322. Amino acid production by this strain was compared to that by the strain M690, as summarized below in Table 5.

TABLE 5
Amounts of homoserine, O-acetylhomoserine, methionine and lysine
produced by strains M690 and M1197

| | | O-acetyl- | | |
|---|---|---|---|---|
| | Homoserine | homoserine | Methionine | Lysine |
| Strain | (mM) | (mM) | (mM) | (mM) |
| M690 | 41.6 | 0.0 | 0.0 | 77.2 |
| M1179 | 26.4 | 1.9 | 0.7 | 79.2 |

The strain M1197 was transformed with DNA F (also referred to as pH399, SEQ ID NO: 27) to yield a “Campbell in” strain, which was subsequently “Campbelled out” to yield strain M1494. This strain contains a mutation in the gene for the homoserine kinase, which results in an amino acid change in the resulting homoserine kinase enzyme from T190 to A190 (referred to as HskT190A). Amino acid production by the strain M1494 was compared to the production by strain M1197, as summarized below in Table 6.

TABLE 6
Amounts of homoserine, O-acetylhomoserine, methionine and lysine
produced by strains M1197 and M1494

| | | O-acetyl- | | |
|---|---|---|---|---|
| | Homoserine | homoserine | Methionine | Lysine |
| Strain | (mM) | (mM) | (mM) | (mM) |
| M1197 | 26.4 | 1.9 | 0.7 | 79.2 |
| M1494 | 18.3 | 0.2 | 2.5 | 50.1 |

The strain M1494 was transformed with DNA D (also referred to as pH484, SEQ ID NO:28) to yield a “Campbell in” strain, which was subsequently “Campbelled out” to yield the M1990 strain. The M1990 strain overexpresses a metY allele using both a groES-promoter and an EFTU (elongation factor Tu)-promoter (referred to as P₄₉₇ P₁₂₈₄ metY). The sequence of P₄₉₇P₁₂₈₄ promoter is set forth in SEQ ID NO:29 Amino acid production by the strain M1494 was compared to the production by strain M1990, as summarized below in Table 7.

TABLE 7
Amounts of homoserine, O-acetylhomoserine, methionine and lysine
produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1494 | 18.3 | 0.2 | 2.5 | 50.1 |
| M1990 | 18.2 | 0.3 | 5.6 | 48.9 |

The strain M1990 was transformed with DNA E (also referred to as pH 491, SEQ ID NO: 30) to yield a “Campbell in” strain, which was then “Campbelled out” to yield a “Campbell out” strain M2014. The M2014 strain overexpresses a metA allele using a superoxide dismutase promoter (referred to as P₃₁₁₉ metA). The sequence of P₃₁₁₉ promoter is set forth in SEQ ID NO: 20. Amino acid production by the strain M2014 was compared to the production by strain M1990, as summarized below in Table 8

TABLE 8
Amounts of homoserine, O-acetylhomoserine, methionine and lysine
produced by strains M1494 and M1990

| | | O-acetyl- | | |
|---|---|---|---|---|
| | Homoserine | homoserine | Methionine | Lysine |
| Strain | (mM) | (mM) | (mM) | (mM) |
| M1990 | 18.2 | 0.3 | 5.6 | 48.9 |
| M2014 | 12.3 | 1.2 | 5.7 | 49.2 |

Experiment 2—Deletion of mcbR from M2014

Plasmid pH429 containing an RXA00655 deletion, (SEQ ID No. 31) was used to introduce the mcbR deletion into C. glutamicum via integration and excision (see WO 2004/050694 A1).

Plasmid pH429 was transformed into the M2014 strain with selection for kanamycin resistance (Campbell in). Using sacB counter-selection, kanamycin-sensitive derivatives of the transformed strain were isolated which presumably had lost the integrated plasmid by excision (Campbell out). The transformed strain produced kanamycin-sensitive derivatives that made small colonies and larger colonies. Colonies of both sizes were screened by PCR to detect the presence of mcbR deletion. None of the larger colonies contained the deletion, whereas 60-70% of the smaller colonies contained the expected mcbR deletion.

When an original isolate was streaked for single colonies on BHI plates, a mixture of tiny and small colonies appeared. When the tiny colonies were restreaked on BHI, once again a mixture of tiny and small colonies appeared. When the small colonies were restreaked on BHI, the colony size was usually small and uniform. Two small single colony isolates, called OM403-4 and OM403-8, were selected for further study.

Shake flask experiments (Table 9) showed that OM403-8 produced at least twice the amount of methionine as the parent M2014. This strain also produced less than one-fifth the amount of lysine as M2014, suggesting a diversion of the carbon flux from aspartate semialdehyde towards homoserine. A third striking difference was a greater than 10-fold increase in the accumulation of isoleucine by OM403 relative to M2014. Cultures were grown for 48 hours in standard molasses medium.

TABLE 9
Amino acid production by isolates of the OM403 strain in
shake flask cultures inoculated with freshly grown cells

| | Colony | Deletion | Met | Lys | Hse + Gly | Ile |
|---|---|---|---|---|---|---|
| Strain | size | ?mcbR | (g/l) | (g/l) | (g/l) | (g/l) |
| M2014 | Large | none | 0.2 | 2.4 | 0.3 | 0.04 |
| | | | 0.2 | 2.5 | 0.3 | 0.03 |
| | | | 0.2 | 2.4 | 0.3 | 0.03 |
| | | | 0.4 | 3.1 | 0.4 | 0.03 |
| OM403-8 | Small | ? RXA0655 | 1.0 | 0.3 | 0.8 | 0.8 |
| | | | 1.0 | 0.3 | 0.8 | 0.8 |
| | | | 0.9 | 0.3 | 0.8 | 0.8 |
| | | | 1.0 | 0.3 | 0.8 | 0.6 |

Also as shown in Table 10, there was a greater than 15-fold decrease in the accumulation of O-acetylhomoserine by OM403 relative to M2014. The most likely explanation for this result is that most of the O-acetylhomoserine that accumulates in M2014 is being converted to methionine, homocysteine, and isoleucine in OM403.

Cultures were grown for 48 hours in standard molasses medium.

TABLE 10
Amino acid production by two isolates of OM403 in shake flask cultures
inoculated with freshly grown cells.

| | Deletion | Met | OAc-Hse | Ile |
|---|---|---|---|---|
| Strain | ?mcbR | (g/l) | (g/l) | (g/l) |
| M2014 | None | 0.4 | 3.4 | 0.1 |
| | | 0.4 | 3.2 | 0.1 |
| OM403-4 | ? RXA0655 | 1.7 | 0.2 | 0.3 |
| | | 1.5 | 0.1 | 0.3 |
| OM403-8 | ? RXA0655 | 2.2 | <0.05 | 0.6 |
| | | 2.5 | <0.05 | 0.6 |

Experiment 3—Decreasing metQ Expression

In order to decrease the import of methionine in OM403-8, the promoter and 5′ portion of the metQ gene were deleted. The metQ gene encodes a subunit of a methionine import complex that is required for the complex to function. This was accomplished using the standard Campbelling in and Campbelling out technique with plasmid pH449 (SEQ ID NO: 32). OM403-8 and OM456-2 were assayed for methionine production in shake flask assays. The results (Table 11) show that OM456-2 produced more methionine than OM403-8. Cultures were grown for 48 hours in standard molasses medium.

TABLE 11
Shake flask assays of OM456-2

| | | [Met] | [Lys] | [Gly/Hse] | [OAcHS] | [Ile] |
|---|---|---|---|---|---|---|
| Strain | vector | (g/l) | (g/l) | (g/l) | (g/l) | (g/l) |
| OM403-8 | none | 4.0 | 0.8 | 2.2 | 0.4 | 1.9 |
| | | 3.9 | 0.6 | 2.2 | 0.4 | 1.9 |
| OM456-2 | none | 4.2 | 0.4 | 2.3 | 0.4 | 2.3 |
| | | 4.3 | 0.5 | 2.4 | 0.4 | 2.3 |

### Experiment 4—Construction of OM469

A strain referred to as OM469 was constructed which included both deletion of metQ and overexpression of metF by replacing the metF promoter with the phage lambda P_{R} promoter in OM456-2. This was accomplished using the standard Campbelling in and Campbelling out technique with plasmid pOM427 (SEQ ID NO 33). Four isolates of OM469 were assayed for methionine production in shake flask culture assays where they all produced more methionine than OM456-2, as shown in Table 12. Cultures were grown for 48 hours in standard molasses medium containing 2 mM threonine.

TABLE 12
Shake flask assays of OM469, a derivative of OM456-2 containing the
phage lambda P_{R} promoter in place of the metF promoter.

| | | metF | | [Met] | [Lys] | [Gly/Hse] | [OAcHS] | [Ile] |
|---|---|---|---|---|---|---|---|---|
| Strain | | promoter | MetQ | (g/l) | (g/l) | (g/l) | (g/l) | (g/l) |
| OM428-2 | | λP_{R} | native | 4.5 | 0.5 | 2.6 | 0.4 | 2.6 |
| | | | | 4.6 | 0.4 | 2.6 | 0.3 | 2.5 |
| OM456-2 | | Native | ΔmetQ | 4.2 | 0.4 | 2.4 | 0.3 | 2.5 |
| | | | | 4.2 | 0.5 | 2.4 | 0.3 | 2.5 |
| OM469 | -1 | λP_{R} | ΔmetQ | 5.0 | 0.5 | 2.7 | 0.4 | 3.1 |
| | -2 | | | 4.9 | 0.5 | 2.7 | 0.4 | 2.8 |
| | -3 | | | 4.8 | 0.4 | 2.6 | 0.4 | 2.7 |
| | -4 | | | 4.7 | 0.5 | 2.6 | 0.4 | 2.8 |

### Experiment 5—Construction of M 2543

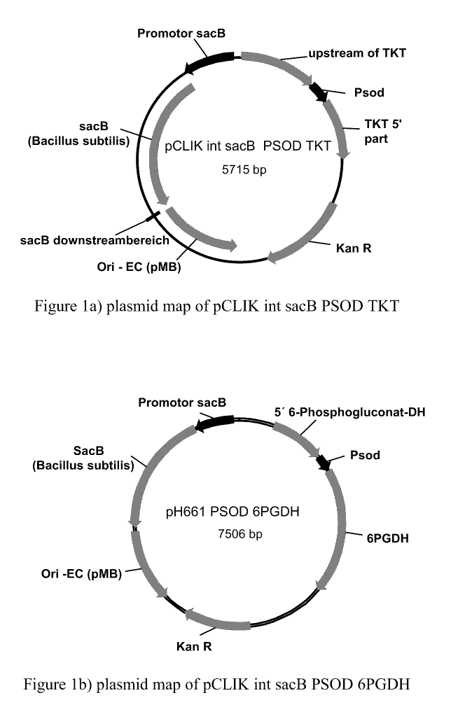

The strain OM469-2 was transformed by electroporation with the plasmid pCLIK5A int sacB PSOD TKT as depicted in SEQ ID NO. 34 (FIG. 1 a)). This was accomplished using the standard Campbelling in and Campbelling out technique.

Isolates of OM 469 PSOD TKT which were labelled M2543 were assayed for methionine production in shake flask culture assays, where they produced more methionine than OM469-2. The results of strain M2543 Are shown in Table 13.

TABLE 13
Shake flask assays of OM469 and M2543

| | | met | | | | | | |
|---|---|---|---|---|---|---|---|---|
| | | genes | | | | | | |
| | plas- | on | [Met] | [Lys] | [Gly] | [Hse] | [AHs] | [Ile] |
| Strain | mid | plasmid | (mM) | (mM) | (mM) | (mM) | (mM) | (mM) |
| OM469-2 | | None | 14 | 3.4 | 16 | 1.7 | 0.3 | 11.8 |
| M2543# | | None | 20.4 | 1.9 | 21.8 | 0.8 | <0.1 | 12.4 |

### Experiment 6—Construction of Strains Containing a Promoter and or Mutations in the 6-Phosphogluconate Dehydrogenase

The strain OM469-2 or M2543 was/were transformed by electroporation with the plasmid pCLIK5A PSODH661 PSOD 6PGDH as depicted in SEQ ID No. 35 (FIG. 1 b). This was accomplished using the standard Campbelling in and Campbelling out technique. The resulting strains contained either only the promoter P_{SOD} or the promotor together with one or two mutations as described in table 14.

Isolates of M2543 PSOD 6PGDH which are labelled GK 1508, 1511 and GK1513 were assayed for methionine production in shake flask culture assays, where they produced more methionine than M2543. The results are shown in Table 14.

TABLE 14
Shake flask assays of OM469 and M2543

| | Promotor | | [Met] |
|---|---|---|---|
| Strain | introduced | Mutation | (mM) |
| M2543 | None | None | 21.6 |
| GK1508 | P_{SOD} | P150S, | 24.6 |
| | | S353F | |
| GK1511 | P_{SOD} | None | 24.7 |
| GK1513 | P_{SOD} | P150S | 25.9 |

1. A method of producing methionine in Coryneform bacteria comprising the step of cultivating the Coryneform bacteria derived by genetic modification from a starting organism such that said Coryneform bacterium displays an increased amount and/or activity of at least two enzymes of the pentose phosphate pathway compared to the starting organism.

2-14. (canceled)

15. The method according to claim 1, wherein at least about 2%, at least about 5%, at least about 10%, at least about 20%, preferably at least about 30%, at least about 40%, at least about 50% and more preferably at least about factor 2, at least about factor 5 and at least about factor 10 more methionine is produced by cultivating the bacterium compared to cultivating the starting organism.

16-30. (canceled)

31. The method according to claim 1, wherein the amount and/or activity of at least transketolase and glucose-6-phosphate-dehydrogenase, transketolase and 6-phospho-gluconate-dehydrogenase, or glucose-6-phosphate-dehydrogenase and 6-phospho-gluconate-dehydrogenase is increased compared to the starting organism.

32. The method according to claim 31, wherein the amount and/or activity of at least transketolase, glucose-6-phosphate-dehydrogenase and 6-phospho-gluconate-dehydrogenase is increased compared to the starting organism.

33. The method according to claim 1, wherein the amount and/or activity of said enzyme(s) is increased by increasing the copy number of the nucleic acid sequences encoding said enzymes, increasing transcription and/or translation of the genes encoding said enzymes, introducing mutations in the nucleic acid sequences encoding said enzymes or a combination thereof.

34. The method according to claim 33, wherein the gene copy number is increased by using autonomously replicating vectors comprising nucleic acid sequence encoding said enzymes and/or by chromosomal integration of additional copies of nucleic acid sequences encoding said enzymes into the genome of the starting organism.

35. The method according to claim 33, wherein transcription is increased by using strong promoter.

36. The method according to claim 35, wherein the strong promoter is selected from the group comprising P_{EFTu}, P_{groES}, P_{SOD} and P_{λR}.

37. The methods according to claim 35, wherein the amount and/or activity of transketolase and 6-phospho-gluconate-dehydrogenase is increased compared to a starting organism by replacing their respective endogenous promoters with a strong promoter which preferably is P_{SOD}.

38. The method according to claim 33, wherein transketolase carries at least one mutation at a position corresponding to position 293 or 327 of SEQ ID No. 12 and wherein 6-phospho-gluconate-dehydrogenase carries at least one mutation at a position corresponding to position 150, 209, 269, 288, 329, 330 or 353 of SEQ ID NO:6.

39. A method according to claim 37, wherein the amount and/or activity of transketolase and 6-phospho-gluconate-dehydrogenase are increased compared to a starting organism by replacing their respective endogenous promoters with a strong promoter which preferably is P_{SOD}, wherein transketolase carries at least one mutation at a position corresponding to position 293 or 327 of SEQ ID No. 12 and wherein 6-phospho-gluconate-dehydrogenase carries at least one mutation at a position corresponding to position 150, 209, 269, 288, 329, 330 or 353 of SEQ ID NO:6.

40. A method according to claim 1, wherein the Coryneform bacterium is selected from the group comprising the species Corynebacterium glutamicum, Corynebacterium acetoglutamicum, Corynebacterium jeikeum, Corynebacterium acetoacidophilum, Corynebacterium thermoaminogenes, Corynebacterium melassecola and Corynebacterium effiziens.

41. The method according to claim 40, in which a strain of C. glutamicum is used.

42. A method according to claim 1, wherein at least about 2%, at least about 5%, at least about 10%, at least about 20%, preferably at least about 30%, at least about 40%, at least about 50% and more preferably at least about factor 2, at least about factor 5 and at least about factor 10 more methionine is produced compared to the starting organism.
